SEQUENCE LISTING (137 sequences)

SEQ ID NO: 1 - DNA, 786 bp, H. influenzae
  CDS (1)...(786): HI-0003
atgatgtataaagcagtatttagtgattttaatggcaccttattaacctctcaacatacaatttcccctcgaactgttgtggtaattaagcgtttaacggcgaatggcattccttttgtgccaatttcggcgcgttctcctttaggtattttgccttattggaaacagcttgaaacgaataatgtacttgttgcatttagtggtgcgcttattttgaaccaaaatctcgaaccaatttatagcgtacaaattgagccaaaagatattttagagattaatactgttttggcggaacatcctttgcttggcgtgaattattatacaaataatgattgccatgcgcgcgatgtagaaaataaatgggtgatttatgaacgcagcgtgaccaaaattgagattcatccttttgatgaagtagcgacacgttcgccacataaaattcaaattattggggaagcagaagaaatcattgagattgaagttcttttaaaggaaaaatttccacatttaagtatttgtcgttcccacgctaattttttagaggtaatgcacaagagtgcaaccaaaggaagtgcggtgcgttttttggaagattattttggcgtacaaactaatgaagtgattgcatttggcgataattttaatgatctggatatgctagaacatgtggggcttggtgttgcaatgggaaatgcgccaaatgaaattaaacaagctgcaaatgtggttacggcaaccaataatgaagatggacttgcattgattttagaagaaaaatttcctgaa

SEQ ID NO: 2 - protein, 262 aa, H. influenzae
MMYKAVFSDFNGTLLTSQHTISPRTVVVIKRLTANGIPFVPISARSPLGILPYWKQLETNNVLVAFSGALILNQNLEPIYSVQIEPKDILEINTVLAEHPLLGVNYYTNNDCHARDVENKWVIYERSVTKIEIHPFDEVATRSPHKIQIIGEAEEIIEIEVLLKEKFPHLSICRSHANFLEVMHKSATKGSAVRFLEDYFGVQTNEVIAFGDNFNDLDMLEHVGLGVAMGNAPNEIKQAANVVTATNNEDGLALILEEKFPE

SEQ ID NO: 3 - DNA, 462 bp, H. influenzae
  CDS (1)...(462): HI-0004
atgggaagtgtattggttgatttgcaaattgccacagaaaatatagagggtttgccaacagaagagcagattgtgcagtgggcaacaggtgctgttcagcctgagggcaatgaagtcgaaatgacggtgcgtattgtggatgaagccgaaagccatgaattaaatttaacttatcgtggaaaagatcgcccgactaacgtgctttcatttccatttgaatgccccgatgaggtggagttgccattgttaggggatctcgttatttgtcgtcaagtcgtggagcgagaagcctcagaacaagagaaaccattaatggcacattgggcgcatatggtcgtgcatggtagcttacatttactcggttatgatcatattgaagacgatgaagcggaagaaatggaaagtttagaaacccaaattatgcaaggattaggctttgatgatccttatctagcagagaaa

SEQ ID NO: 4 - protein, 154 aa, H. influenzae
MGSVLVDLQIATENIEGLPTEEQIVQWATGAVQPEGNEVEMTVRIVDEAESHELNLTYRGKDRPTNVLSFPFECPDEVELPLLGDLVICRQVVEREASEQEKPLMAHWAHMVVHGSLHLLGYDHIEDDEAEEMESLETQIMQGLGFDDPYLAEK

SEQ ID NO: 5 - DNA, 810 bp, H. influenzae
  CDS (1)...(810): HI-0005
atgcagtgctggattcgacaaactatcaacttttttagaaagacaaaaaatacagaaaaattgaccgcacttttgcaacaaaaagaggacatactggctgttgagataccagtttctttggtatataacggcatttctcacgcagttatgatgtgttccccaaacaatttagaggattttgctttaggcttttctctaacagagggaattatcaataaaccagaagatatttatggtattgatgttgtagaagtttgcaatggtattgaagtgcaaattgaactttccagccgtaaatttatggcattaaaagagcatcgtcgtaatcttacggggcgtactggttgtggtatttgtggtacagaacaattgaatcaagtatataaaacttttccgaaattagactgtacttttcaatttgatttaaatttgctagatagttgtcttattgatcttcaaaaaaatcaattgcttggatgtaaaactggtgctacccatgcttgtgcatttttcgatttatatggaagtatgttagctatttacgaagatgtgggtcgccacgttgcattagataaattgcttggttggcacgcaaaatcgggtaaacctagaggttttattttagcctccagtcgagcgagttatgaaatggtgcaaaaaacagtggcttgcggggtggaaatgttagtcacgatttctgccgcaacggatttagcagtaacaatggcagaaaaacataatttgactttaattggttttgctagggaaggaaaaggaaatatttatagcggacatctacgacttcacaat

SEQ ID NO: 6 - protein, 270 aa, H. influenzae
MQCWIRQTINFFRKTKNTEKLTALLQQKEDILAVEIPVSLVYNGISHAVMMCSPNNLEDFALGFSLTEGIINKPEDIYGIDVVEVCNGIEVQIELSSRKFMALKEHRRNLTGRTGCGICGTEQLNQVYKTFPKLDCTFQFDLNLLDSCLIDLQKNQLLGCKTGATHACAFFDLYGSMLAIYEDVGRHVALDKLLGWHAKSGKPRGFILASSRASYEMVQKTVACGVEMLVTISAATDLAVTMAEKHNLTLIGFAREGKGNIYSGHLRLHN

SEQ ID NO: 7 - DNA, 402 bp, H. influenzae
  CDS (1)...(402): HI-0011
atgaacagacgcgatcttcttttacaagaaatgggcatttcccagtgggaattatatcgccccgaggtactgcaaggttcagtaggaattagtgtggcagagaatattcgccttatcactgtttccgatgaaaatatcagtagctcgcctttgttggctgatgtgctgttaagccttaatcttaaaaaagaaaattgtttatgtttgaattacgatcaaatccagcatatggaatgtaaacagcctattcgttattggttactatcagaaaatagcgaccaaattgaccgcactttgccattttgcaagcaggctgagcaggtttatcgctcgccaagttggcagcaatttcaatctaatcatcaagccaaacgagcgttgtggcaacaaattcagcagcct

SEQ ID NO: 8 - protein, 134 aa, H. influenzae
MNRRDLLLQEMGISQWELYRPEVLQGSVGISVAENIRLITVSDENISSSPLLADVLLSLNLKKENCLCLNYDQIQHMECKQPIRYWLLSENSDQIDRTLPFCKQAEQVYRSPSWQQFQSNHQAKRALWQQIQQP

SEQ ID NO: 9 - DNA, 1047 bp, H. influenzae
  CDS (1)...(1047): HI-0015
atgtcaaatttattttttgtgattttattggctgtcggctttggtgtgtggaaagttttagattattttcagttaccaaatacttttagtattttgttactaattttgaccgcactttctggcgtattatggtgttatcatcgttttgtggtgctgccaaaacgtcatcgtcaagtggcacgtgcagaacaacgttctggtaaaaccttaagtgaggaagaaaaagccaaaattgaaccgatttctgaggcttcagaatttttgtcttcactttttcctgtgcttgcagtggtatttttggttcgttcttttttgtttgaaccgtttcaaattccctctggctcaatggagtccactttacgcgttggcgattttttagttgtgaataaatatgcttatggtgtgaaagatccgattttccaaaacaccattattgcaggcgaaaaaccacaacgtggcgatgtgattgtgtttaaagcaccacaacaagcgttaattcgtactggtcttggggcaacacgagcggcttttgcagaaaatttagcgttaagttcaaaagataatatgtctggtgtggattatattaagcgtattgttggaaagggcggagatcgcgtcatttttgatgtggaacaaaaaacgctaaaagtggtatatggtaaagaggggaaaccctgtgaaattgattgcgaaactaaggcgtttgaatatacacaaaatccaacaaatcctgcttttccaaatgaattagaattgactgaaaaaggcgatgtaacacataacgtgttaattagtgagtatcgtcgttattcagaccttgaatttttcccacaagagggaatgcaaactgcagaatggcttgtgccagaggggcagtattttgtgatgggggatcatcgcgatcacagcgatgacagtcgtttctggggctttgtacctgaaaaaaatattgtgggtaaagccacttatatttggatgagcttagaaaaagaagcgaatgaatggccaacaggtttccgttttgagcgtttctttacagcaataaaa

SEQ ID NO: 10 - protein, 349 aa, H. influenzae
MSNLFFVILLAVGFGVWKVLDYFQLPNTFSILLLILTALSGVLWCYHRFVVLPKRHRQVARAEQRSGKTLSEEEKAKIEPISEASEFLSSLFPVLAVVFLVRSFLFEPFQIPSGSMESTLRVGDFLVVNKYAYGVKDPIFQNTIIAGEKPQRGDVIVFKAPQQALIRTGLGATRAAFAENLALSSKDNMSGVDYIKRIVGKGGDRVIFDVEQKTLKVVYGKEGKPCEIDCETKAFEYTQNPTNPAFPNELELTEKGDVTHNVLISEYRRYSDLEFFPQEGMQTAEWLVPEGQYFVMGDHRDHSDDSRFWGFVPEKNIVGKATYIWMSLEKEANEWPTGFRFERFFTAIK

SEQ ID NO: 11 - DNA, 381 bp, H. influenzae
  CDS (1)...(381): HI-0017
atgattaaaggtattcaaattactcaagcagcaaatgacaatttgcttaattcattctggttattagacagcgaaaaaaatgaagcacgttgcttatgtgcaaaaggcgaattcgctgaagaccaagttgttgcagttagcgaattaggtcaaatcgaataccgtgaattaccagtaaacgtagcaccaactgttaaagttgaaggtggccaacacttaaacgtaaacgtattacgtcgtgaaacgttagaagatgcggtaaacaatccagataaatatccacaattaactatccgtgtttctggttacgcagtacgtttcaactctttaacgccagaacaacaacgcgacgtgatcactcgtactttcactgaaagccta

SEQ ID NO: 12 - protein, 127 aa, H. influenzae
MIKGIQITQAANDNLLNSFWLLDSEKNEARCLCAKGEFAEDQVVAVSELGQIEYRELPVNVAPTVKVEGGQHLNVNVLRRETLEDAVNNPDKYPQLTIRVSGYAVRFNSLTPEQQRDVITRTFTESL

SEQ ID NO: 13 - DNA, 657 bp, H. influenzae
  CDS (1)...(657): HI-0018
atgaaaaactggacagacgttatcggaacagaaaaagcgcaaccttactttcaacacacactacaacaggttcatcttgcaagagcaagcgggaaaacgatttatcccccacaagaagatgtatttaacgcattcaaatatactgcttttgaggatgtaaaagtggtaattttaggtcaggatccttatcatggaccaaaccaagcgcacggcttggctttttcagtaaaacctgaagtagccattcccccttccctattaaatatatataaagaactcacacaagatatttcgggatttcaaatgccatcaaatggttatttagtcaaatgggcagaacaaggggtattgctacttaacactgtgcttaccgtggaacgaggtatggcacattcacacgccaatttaggttgggaaaggtttacagataaagttattgcagtactcaatgaacatcgtgaaaaactggtgtttttactttggggcagtcacgcacaaaaaaaagggcaaatgattgaccgcactcgtcaccttgttttaacggctccgcatccttccccgttgtcagcacatcgaggtttctttggttgtcgtcatttttccaaaacaaattcatatttggaaagccacggaataaaaccgatagattggcaaatc

SEQ ID NO: 14 - protein, 219 aa, H. influenzae
MKNWTDVIGTEKAQPYFQHTLQQVHLARASGKTIYPPQEDVFNAFKYTAFEDVKVVILGQDPYHGPNQAHGLAFSVKPEVAIPPSLLNIYKELTQDISGFQMPSNGYLVKWAEQGVLLLNTVLTVERGMAHSHANLGWERFTDKVIAVLNEHREKLVFLLWGSHAQKKGQMIDRTRHLVLTAPHPSPLSAHRGFFGCRHFSKTNSYLESHGIKPIDWQI

SEQ ID NO: 15 - DNA, 960 bp, H. influenzae
  CDS (1)...(960): HI-0026
atgtctacgcctttcaaaatggaacgcggtgtaaaatatcgcgatgcggcaaaaacctcaattattcccgtaaaaaatatcgatcctaatcaagatttattaaaaaaaccagaatggatgaaaattaaacttccagcaagttcagctaaaattgaaagcattaaaaatggaatgcgccgtcacggattacattctgtttgtgaggaagcatcttgtccaaatttacacgaatgttttaatcatggcacagccacttttatgattttaggtgcaatttgtacgcgccgttgcccattctgtgatgtggctcacggtaaaccattaccgccagatcctgaagagccacaaaaattggcagaaacgattcaagatatgaaacttaaatatgtggtaatcacatcggtggatcgtgatgacttgcctgatcgtggtgctggtcatttttctgaatgtgtaaaagctgtgcgcgagcttaatcctaacattaagattgaaattttagtgcctgattttcgtggtcgcattacacaggctttagagaaattaaaagataatccgccagatgtctttaaccataatttagaaaacgtaccgcgcttatataaagaaattcgtccaggtgcggattacgaatggtcacttaaattattgcgtgaattcaaagaaatattcccaaatatcccaaccaaatctggtttaatggttgggttaggcgaaaccaacgaggaaattttgcaagtgatgcaagatttacgtgataacggtgtgactatgctcacgttgggtcaatatcttcaacctagccgccatcatttgcctgttgcccgttatgtgccgccaactgaatttgatgaattccgtgataaagccaatgaaatgggctttgaacatgcggcttgtgggccatttgttcgttcttcttatcacgctgatttacaagccagtggcggattagtgaaa

SEQ ID NO: 16 - protein, 320 aa, H. influenzae
MSTPFKMERGVKYRDAAKTSIIPVKNIDPNQDLLKKPEWMKIKLPASSAKIESIKNGMRRHGLHSVCEEASCPNLHECFNHGTATFMILGAICTRRCPFCDVAHGKPLPPDPEEPQKLAETIQDMKLKYVVITSVDRDDLPDRGAGHFSECVKAVRELNPNIKIEILVPDFRGRITQALEKLKDNPPDVFNHNLENVPRLYKEIRPGADYEWSLKLLREFKEIFPNIPTKSGLMVGLGETNEEILQVMQDLRDNGVTMLTLGQYLQPSRHHLPVARYVPPTEFDEFRDKANEMGFEHAACGPFVRSSYHADLQASGGLVK

SEQ ID NO: 17 - DNA, 276 bp, H. influenzae
  CDS (1)...(276): HI-0028
atgacaatagaaaacgattatgcaaaactcaaggaattaatggaatttccagcaaaaatgacttttaaagttgcgggaattaatcgagaagggttagcacaagatcttatacaagtggtgcaaaaatatattaaaggcgattatattccaaaagaaaagcgcagtagtaaaggcacttataattcagtttcaattgatattattgctgaaaactttgatcaggtagaaacactctataaagaactggctaaagttgaaggcgtgaaaatggttatt

SEQ ID NO: 18 - protein, 92 aa, H. influenzae
MTIENDYAKLKELMEFPAKMTFKVAGINREGLAQDLIQVVQKYIKGDYIPKEKRSSKGTYNSVSIDIIAENFDQVETLYKELAKVEGVKMVI

SEQ ID NO: 19 - DNA, 465 bp, H. influenzae
  CDS (1)...(465): HI-0033
gtgaaaatcacgttaattgccgtcggaacaaaaatgccttcttgggttacaacgggttttgaggaatatcaacgccgtttcccaaaagatatgccttttgaactaattgaaatcccagcaggcaagcgtggaaaaaatgctgatattaaacgcattttagaacaagaaggtaaagcgatgttagcggcctgtggaaaaggcaaagtcgtgacgttagatattcctggtaaaccttggacgacgccgcagctagctgagcaactagaagcgtggaaaaatgatggtcgcgatgtttgtttattgattggtgggcctgaggggctttcgccagaatgcaaagctgctgcagagcaaagttggtcgctttctcccttgacattacctcacccgcttgttcgtgtcgtggtggctgaaagtttgtatcgtgcgtggtcgctaaccactaatcatccttatcatcgagaa

SEQ ID NO: 20 - protein, 155 aa, H. influenzae
VKITLIAVGTKMPSWVTTGFEEYQRRFPKDMPFELIEIPAGKRGKNADIKRILEQEGKAMLAACGKGKVVTLDIPGKPWTTPQLAEQLEAWKNDGRDVCLLIGGPEGLSPECKAAAEQSWSLSPLTLPHPLVRVVVAESLYRAWSLTTNHPYHRE

SEQ ID NO: 21 - DNA, 495 bp, H. influenzae
  CDS (1)...(495): HI-0051
atgggggataaagaaggagcttgttttatgaaaatagcaaagtatttagacaaagcattagaatatctttctatcttggcattagttattatgatttctctcgtgttttttaattctgtcttgaggtacttctttgattctggaattgcattttctgaagagttttctaggatttgttttgtatatatgatttcctttgggattatacttgttgccaaggataaagctcatcttactgttgatattattattcctgcgttgcctgagcagtatagaaaaatagtcttaatagttgcaaatatatgcgttctgattgcaatgatttttatagcttatggtgcattacaactgatgtctttgacctatactcaacaaatgccagctacaggtatatcttcatcttttttatatttagctgctgttatatctgccgtatcttacttttttattgtcatgtttagcatgattaaagactataaagaatcctctgataaa

SEQ ID NO: 22 - protein, 165 aa, H. influenzae
MGDKEGACFMKIAKYLDKALEYLSILALVIMISLVFFNSVLRYFFDSGIAFSEEFSRICFVYMISFGIILVAKDKAHLTVDIIIPALPEQYRKIVLIVANICVLIAMIFIAYGALQLMSLTYTQQMPATGISSSFLYLAAVISAVSYFFIVMFSMIKDYKESSDK

SEQ ID NO: 23 - DNA, 996 bp, H. influenzae
  CDS (1)...(996): HI-0059
atgcccttctggtattccaactccaaacttatttggctcttatcgcctttttctttattgttttggttgattagccaacttcgtcgcgccttattctctttggggctgaagtcttcttatcgcgcaccaaaaccagtgataattgtgggaaatttgtctgtgggtggaaatggcaaaacgcctgtggttgtttggcttatggaagaattaaaaaaacgaggtctgcgtgtaggtgtgatttctcgtggttacggcagtaaatctaaaacttatccgttattcgtcactaaaaatacaaatccaattgaaggtggcgatgagcctgtattgatcgctaaacgtactaatgcgccagttgtgatttccccgaatcgccagcaagcgattgaattactcttaagccaagcagagtgcgatattattatttctgatgatggtttgcagcattatcaattacaacgtgatttagaaattgtcgtaatggacgctgagcgcgcattgggaaatggttttgtattgccagcaggtccattgcgtgaattaccaagtcgattaaaatctgtcgattttgtgatcactaatggtggaaaaaatcagtattcagatgcagttatgcgtcttgtgcctcatttcgcgattaatttaaaaaccaatgaaaaacgccaattaaatgaatttcaatctggtgttgccatcgcagggattggcaatccacagcgtttttttactatgttagaaaagttagggattcagttaaagcaaactcaagcatttcaagatcatcaacattttgaagcgtctcaattagaaaaacttgctgaaaatcaaccgctctttatgacggaaaaagatgccgtaaaatgccaatcttttgctaaagataattggtggtatgtccctgtggatgcggagattattgaggctgaaaaacaacgtgaaaatttaccgcacttttgggccaaaatagacaaacttgtggagcaatacagaaatggc

SEQ ID NO: 24 - protein, 332 aa, H. influenzae
MPFWYSNSKLIWLLSPFSLLFWLISQLRRALFSLGLKSSYRAPKPVIIVGNLSVGGNGKTPVVVWLMEELKKRGLRVGVISRGYGSKSKTYPLFVTKNTNPIEGGDEPVLIAKRTNAPVVISPNRQQAIELLLSQAECDIIISDDGLQHYQLQRDLEIVVMDAERALGNGFVLPAGPLRELPSRLKSVDFVITNGGKNQYSDAVMRLVPHFAINLKTNEKRQLNEFQSGVAIAGIGNPQRFFTMLEKLGIQLKQTQAFQDHQHFEASQLEKLAENQPLFMTEKDAVKCQSFAKDNWWYVPVDAEIIEAEKQRENLPHFWAKIDKLVEQYRNG

SEQ ID NO: 25 - DNA, 1761 bp, H. influenzae
  CDS (1)...(1761): HI-0060
atgcaagagcaaaaattacaagaaaatgatttttcaaccttacaaacgtttaagcgtttgtggccaatgattaaaccttttaaagcggggcttattgtttctggcgtcgcgttagtttttaatgcattagctgactcaggcttgatttatttgttaaaaccgttgttggacgatggttttggtaaggcaaaccattcatttttgaaaatgatggcttttgtcgtcgttgggatgattattttacgcggcattaccaactttatttctaattattgcttggcgtgggtatcaggcaaagttgtcatgacaatgcgtcgccgcttgtttaaacatttaatgtttatgccagtgagtttctttgatcaaaattcaacagggcgtttactttctcggattacttatgattcccaaatgattgcaagttcttcttcaggatctttgattacaattgtgcgagaaggagcatatattatttcgctattcgcagtgatgttttataccagttgggaattaacaattgtgctttttattataggtccaatcattgctgttttaattcgtttggtatcaaaaatttttcgtagattaagtaagaacttacaagattcaatgggtgaacttacgtctgctacagaacagatgttaaaaggtcataaagttgtgctttctttcggagggcaacatgtggaagaagtgcattttaatcatgttagtaatgatatgcgtcgaaaaagcatgaaaatggtgacagcaaattccatttctgatcctgtggtgcaagttattgcatctctcgcattagctacggtgctctatttagctaccactccattgattgcagaagataatttgagtgcaggctcatttacagtggtattttcttcaatgttagctatgatgcgtccgttaaaatcattaactgcggtgaatgcacaatttcaaagtggaatggcagcatgtcaaacgctatttgccattttagatttagagccagaaaaagatgacggggcttataaagcagaacctgcgaaaggcgagttagaatttaaaaatgtgagttttgcatatcaaggaaaagatgaacttgcattaaataatatttcttttagcgttccagctggaaaaaccgtagctctagtgggacgttctggatcgggcaaatcaaccattgctaatttagtgacacgtttttacgatattgagcaaggcgaaattttactggatggcgtaaatatccaagattatcgtttatctaatttacgtgaaaactgcgctgtggtttctcaacaagtccatttatttaacgatactattgcgaataatattgcttatgcagcacaagataagtattctcgcgaagaaattatcgctgcagcaaaagcggcttatgctttagagtttatcgaaaaattaccgcaagtttttgatacggtaattggcgaaaatggcactagcctatcaggtgggcaacgtcagcgtttagcgattgctcgtgctttattgcgtaattcgccagtattaattttagatgaagctacatctgcactagatacggaatcagaacgagcaatccaatctgcattagaggaattaaagaaagatcgcacggttgttgtgattgcccatagattatctactattgaaaacgcggatgaaattcttgtgattgatcacggagaaattcgtgagcgtggcaaccataaaacattgcttgaacaaaatggtgcctataaacagttgcatagtatgcagtttactggc

SEQ ID NO: 26 - protein, 587 aa, H. influenzae
MQEQKLQENDFSTLQTFKRLWPMIKPFKAGLIVSGVALVFNALADSGLIYLLKPLLDDGFGKANHSFLKMMAFVVVGMIILRGITNFISNYCLAWVSGKVVMTMRRRLFKHLMFMPVSFFDQNSTGRLLSRITYDSQMIASSSSGSLITIVREGAYIISLFAVMFYTSWELTIVLFIIGPIIAVLIRLVSKIFRRLSKNLQDSMGELTSATEQMLKGHKVVLSFGGQHVEEVHFNHVSNDMRRKSMKMVTANSISDPVVQVIASLALATVLYLATTPLIAEDNLSAGSFTVVFSSMLAMMRPLKSLTAVNAQFQSGMAACQTLFAILDLEPEKDDGAYKAEPAKGELEFKNVSFAYQGKDELALNNISFSVPAGKTVALVGRSGSGKSTIANLVTRFYDIEQGEILLDGVNIQDYRLSNLRENCAVVSQQVHLFNDTIANNIAYAAQDKYSREEIIAAAKAAYALEFIEKLPQVFDTVIGENGTSLSGGQRQRLAIARALLRNSPVLILDEATSALDTESERAIQSALEELKKDRTVVVIAHRLSTIENADEILVIDHGEIRERGNHKTLLEQNGAYKQLHSMQFTG

SEQ ID NO: 27 - DNA, 2364 bp, H. influenzae
  CDS (1)...(2364): HI-0061
atgaaattaaacttaataactttagttgtcttgttaattgtcgcggatttaacgttgttatttctaccgcaaccgttgctattgccttggcaagttgctctcgttattgcgcttgttttgatttttctttttattttcttgcgtagaaatttcttagttagccttgctttttttgttgcctctcttggctattttcattattcggctttgagtttatcacaacaagctcaaaatattaccgctcaaaagcaagtggtaacttttaagattcaagaaattttgcaccaacaggattatcaaacgcttatcgccacagcaacattggagaataatttgcaagaacaacgaattttcttaaattggaaagcgaaagaggtgcctcaattatcggaaatttggcaagctgaaatttctttacgttccctttctgcacgattaaatttcggtgggtttgatcggcaacaatggtatttttcaaaaggaattacggctgttggaacggtaaaaagtgcggtgaaaattgcggatgtttcatcattgcgtgcagaaaaattgcaacaagtaaagaagcaaacggaaggattatctctacaaggtttattgattgccttagcttttggcgaacgggcttggttagataaaaccacttggtcaatttaccaacaaaccaataccgcacatcttattgctatttctggcttacatattgggttggctatgggaattggattttgcttggcgcgtgttgtgcaagtgttctttcctacgcgttttattcatccttattttcctttagtttttggtgttttatttgctttaatttatgcgtatttggctggttttagtgtgccaacttttcgtgccatttcagcacttgttttcgttttatttattcaaataatgaggcgacattattcgcccattcagttttttacgttggttgtcggattcttgcttttctgcgatccattaatgccgctttcggtcagtttttggctttcttgtggagcggttgggtgtttgctcctctggtatcgttatgtgcctttttcactttttcaatggaaaaatcgccctttttctccaaaagtgcggtggatttttagtttatttcatttgcaatttgggttattgctcttttttacacctttgcaactttttctatttaatggcttatcgttgagtggatttttagccaattttatggcggttccaatttatagttttttgcttgtgccattaattttatttgccgtttttactaacggcacaatgttttcttggcaactagcaaacaagctagccgaaggaattactgggttaatttctgtttttcaagggaattggctcacggtttcatttaatttagcattgggtttaaccgcactttgtgcaggaatttttatgttaattatttggaatatttaccgagaacccgagatttcatcatcaaactggcaaataaaacgagcaaaattttttacattaaatctcagtaagcctttgctaaaaaatgaacgaatcaacgttttgcgatgttctttcggcattatcttactgtgttttacgattttgttgtttaaacaattgagcaagccaacttggcaggtagatactttagatgtggggcaggggttagctacgttgattgtgaaaaatggtaaagggattctttatgatacgggttcttcttggcgaggtggaagtatggctgagttggaaattttgccttatttacaaagagaagggattgttttggaaaaattgattttaagccacgacgataacgatcacgcaggtggtgcttcgacaattttaaaggcgtatcccaatgtggaattgattaccccttcacggaaaaactatggggaaaattaccgcactttttgtactgctgggcgtgattggcattggcaagggttgcattttcaaatactttctcctcacaacgttgtgacacgagctgataattcccattcttgtgtgattttagtcgatgatggaaagaatagcgttttgctaactggcgatgctgaagcaaaaaatgagcaaatttttgcccgcactttaggcaaaatcgatgtgttgcaagtggggcatcatgggagtaaaacatcgacaagtgaatacttgctttctcaggttagaccagatgtagcgattatttctagtgggcgttggaatccgtggaaattccctcattattcggttatggaaaggcttcatcgctataaaagtgcggtagaaaataccgctgtttcggggcaagtgcgggtaaatttttttcaagaccgattagaaatccagcaagctcgcacaaaattttccccttggtatgcgcgtgtaattggattatcaaaggaa

SEQ ID NO: 28 - protein, 788 aa, H. influenzae
MKLNLITLVVLLIVADLTLLFLPQPLLLPWQVALVIALVLIFLFIFLRRNFLVSLAFFVASLGYFHYSALSLSQQAQNITAQKQVVTFKIQEILHQQDYQTLIATATLENNLQEQRIFLNWKAKEVPQLSEIWQAEISLRSLSARLNFGGFDRQQWYFSKGITAVGTVKSAVKIADVSSLRAEKLQQVKKQTEGLSLQGLLIALAFGERAWLDKTTWSIYQQTNTAHLIAISGLHIGLAMGIGFCLARVVQVFFPTRFIHPYFPLVFGVLFALIYAYLAGFSVPTFRAISALVFVLFIQIMRRHYSPIQFFTLVVGFLLFCDPLMPLSVSFWLSCGAVGCLLLWYRYVPFSLFQWKNRPFSPKVRWIFSLFHLQFGLLLFFTPLQLFLFNGLSLSGFLANFMAVPIYSFLLVPLILFAVFTNGTMFSWQLANKLAEGITGLISVFQGNWLTVSFNLALGLTALCAGIFMLIIWNIYREPEISSSNWQIKRAKFFTLNLSKPLLKNERINVLRCSFGIILLCFTILLFKQLSKPTWQVDTLDVGQGLATLIVKNGKGILYDTGSSWRGGSMAELEILPYLQREGIVLEKLILSHDDNDHAGGASTILKAYPNVELITPSRKNYGENYRTFCTAGRDWHWQGLHFQILSPHNVVTRADNSHSCVILVDDGKNSVLLTGDAEAKNEQIFARTLGKIDVLQVGHHGSKTSTSEYLLSQVRPDVAIISSGRWNPWKFPHYSVMERLHRYKSAVENTAVSGQVRVNFFQDRLEIQQARTKFSPWYARVIGLSKE

SEQ ID NO: 29 - DNA, 474 bp, H. influenzae
  CDS (1)...(474): HI-0065
atggaaagtttgactcaatatatccctgatgaattttctatgctccgcttcggcaaaaaatttgccgaaattcttttaaaattgcatacagaaaaagcaattatggtttatcttaacggtgatcttggggcaggaaaaacaacgctaacgcgcggaatgttgcaaggtatcggtcatcaaggtaatgtaaaaagcccaacttatacgctggttgaagaatacaatattgcaggcaaaatgatttatcattttgatttatatcgtttagcagatcctgaagagctcgaatttatgggcattagagattattttaatacggatagcatctgcttaattgaatggtctgaaaaaggtcaaggcatattgccagaagcggatattttagtgaatattgattattacgatgatgcacgaaatattgaattaatcgcacaaacaaatttgggtaaaaatattataagcgcattttctaat

SEQ ID NO: 30 - protein, 158 aa, H. influenzae
MESLTQYIPDEFSMLRFGKKFAEILLKLHTEKAIMVYLNGDLGAGKTTLTRGMLQGIGHQGNVKSPTYTLVEEYNIAGKMIYHFDLYRLADPEELEFMGIRDYFNTDSICLIEWSEKGQGILPEADILVNIDYYDDARNIELIAQTNLGKNIISAFSN

SEQ ID NO: 31 - DNA, 933 bp, H. influenzae
  CDS (1)...(933): HI-0068
atgaaaccaaccgcaatttttttaatgggcccaacagcctcaggtaaaacagatttagccattcaactacgaagccaacttccagtcgaagtgattagtgtggattcagctcttatttataagggaatggatattggcacagcgaaaccatcaaaagaagaacttgcccttgcgcctcaccgtttaattgatattttagatccatcagaaagctattccgcaatgaatttccgcgatgatgcgctacgcgaaatggcggatattaccgcacaaggcaaaattccgttactagtgggcggcacgatgttgtattacaaagccttaatcgaagggctttcgcccttgccttctgctgatgaaaatattcgcgccgagcttgaacaaaaagcagctcaacaaggttgggcagcattgcatacagaactcgccaaaatcgatcccatttctgccgcccgaattaatcccagtgattctcaacgaatcaatcgcgctttagaagttttttacatcacaggaaaatcactcacagagctgactgaagaaaaaggcgaagccttgccttatgattttgtgcaatttgcaattgcgccgcaagatcgccatgttttgcatgaacgcatcgaacaacgctttcataaaatgattgaactaggctttcaagcagaagtggaaaaactttatgcgcgtggcgatttaaatattaatctgccgtcaattcgctgtgtaggctatcgtcaaatgtgggaatatttgcaaggtgattacgcttacgaggaaatgattttccgtggtatttgcgctacgcgccaacttgcaaaacgccagcttacttggctgcgtggttggaaaacaccaatacaatggctagatagtttacaacctcaacaagcgaaagaaactgtattacgtcacttagattcttatcaaaaaggt

SEQ ID NO: 32 - protein, 311 aa, H. influenzae
MKPTAIFLMGPTASGKTDLAIQLRSQLPVEVISVDSALIYKGMDIGTAKPSKEELALAPHRLIDILDPSESYSAMNFRDDALREMADITAQGKIPLLVGGTMLYYKALIEGLSPLPSADENIRAELEQKAAQQGWAALHTELAKIDPISAARINPSDSQRINRALEVFYITGKSLTELTEEKGEALPYDFVQFAIAPQDRHVLHERIEQRFHKMIELGFQAEVEKLYARGDLNINLPSIRCVGYRQMWEYLQGDYAYEEMIFRGICATRQLAKRQLTWLRGWKTPIQWLDSLQPQQAKETVLRHLDSYQKG

SEQ ID NO: 33 - DNA, 783 bp, H. influenzae
  CDS (1)...(783): HI-0072
atgcacaaaaacttatttcattggttaatggaacgtggttatcaagtgttggtggaaaaagaagtcgccataacacttgagttaccttttgaacatcttgctacgttagaagaaataggccaccgagcccaattagccattgtgattggtggagacggcaatatgctagggcgcgctcgcgtattagcaaaatatgatattccattgattggtattaatcgtggtaatttgggatttttaacggatattgacccgaaaaatgcctattcccagcttgaagcttgtttagaacgtggcgaattttttgtggaagaacgttttttattggaagcaaaaatcgaacgagcaagtgaaatcgtatcaaccagcaatgcggtaaatgaagcggttattcatcccgccaaaattgcacatatgattgattttcacgtatatatcaatgataagtttgcattttctcaacgttctgatggattaattgtttctactccaacaggttctacggcttattctctttccgctggtggacctattttgacaccaaaccttaatgccattgcattagtgccaatgtttccacatacattaacttctcgccctcttgttgttgatggggatagtaaaatatcgattcgttttgctgaacataatacctctcaattagaagtgggctgtgatagtcaaattaccttaccttttaccccagatgatgtggtgcatattcaaaaaagcgagcataaactccgattgcttcatctgaaaattataattattacaatgtgt

SEQ ID NO: 34 - protein, 261 aa, H. influenzae
MHKNLFHWLMERGYQVLVEKEVAITLELPFEHLATLEEIGHRAQLAIVIGGDGNMLGRARVLAKYDIPLIGINRGNLGFLTDIDPKNAYSQLEACLERGEFFVEERFLLEAKIERASEIVSTSNAVNEAVIHPAKIAHMIDFHVYINDKFAFSQRSDGLIVSTPTGSTAYSLSAGGPILTPNLNAIALVPMFPHTLTSRPLVVDGDSKISIRFAEHNTSQLEVGCDSQITLPFTPDDVVHIQKSEHKLRLLHLKIIIITMC

SEQ ID NO: 35 - DNA, 342 bp, H. influenzae
  CDS (1)...(342): HI-0073
atgactagttttgcacagcttgatataaaatctgaagaattggcgattgtgaaaactattttacaacaattagtacctgattataccgtgtgggctttcggttctcgtgtaaagggaaaagcaaaaaaatattccgatcttgaccttgcgattatctctgaggaacctttagattttttagctcgtgacagattaaaggaagctttttctgaatcagatttaccgtggcgcgttgatcttctagattgggctacaacgagcgaagattttagggaaattatccgtaaagtttatgtagttattcaggaaaaagaaaaaacggtcgaaaaaccgaccgctctt

SEQ ID NO: 36 - protein, 114 aa, H. influenzae
MTSFAQLDIKSEELAIVKTILQQLVPDYTVWAFGSRVKGKAKKYSDLDLAIISEEPLDFLARDRLKEAFSESDLPWRVDLLDWATTSEDFREIIRKVYVVIQEKEKTVEKPTAL

SEQ ID NO: 37 - DNA, 438 bp, H. influenzae
  CDS (1)...(438): HI-0074
atgatgactgataagctaaatttaaatgtattagatgctgcattttattcgttagagcaaaccgtagtacaaatttcagacagaaattggtttgatatgcaaccctctattgtacaagataccttaattgcaggtgcaattcagaaatttgaatttgtttatgagttaagtttaaaaatgatgaaacgccaacttcaacaagatgcaattaataccgatgacattggggcttatggatttaaggatattttgcgagaagcattgagatttggtttgattggagatatgtcaaaatgggttgcttatcgtgatatgcgtaatattacatcacatacttatgatcaggaaaaagccatggctgtttatgcacaaattgatgattttttaatagaaagtagtttcctattggaacaattacgtcagagaaatcaatatgac

SEQ ID NO: 38 - protein, 146 aa, H. influenzae
MMTDKLNLNVLDAAFYSLEQTVVQISDRNWFDMQPSIVQDTLIAGAIQKFEFVYELSLKMMKRQLQQDAINTDDIGAYGFKDILREALRFGLIGDMSKWVAYRDMRNITSHTYDQEKAMAVYAQIDDFLIESSFLLEQLRQRNQYD

SEQ ID NO: 39 - DNA, 786 bp, H. influenzae
  CDS (1)...(786): HI-0081
atgcacttcttcgacactcacacgcatcttaattatctccaacaatttactggggaaccgttgtcacagcttattgataatgctaagcaagccgatgtgcaaaaaatactggttgtggcggtgaaagaagctgattttaaaacaatccaaaatatgaccgcactttttcctgataatttgtgctatgggcttgggctccatcctctttatattcaagaacacgccgaaaatgatttgattctgttagaacaagccttaaaaaatcgtgatacaaattgcacggcagtggcagaaattggtttggaacgcgcgattcccgatttgctcaccgatgaactttgggcaaaacaatgtcatttttttgaaagccaactttatttggcaaaacaatttaatttacccgtcaatattcacagtcgaaaaacccatgatcaaatttttacttttttaaaacgtattccattatctaaacttggcgtggtacatggtttttcaggaagttacgatcaagcaaaacgctttgttgatttaggctataaaatcggagttggcggcactatcacttatgaacgagccaataaaactcgtcaagcaatcgcgaagttgccgcttgatgccttagtgttggaaacagacagcccagatatgcctgtatttggttttcaaggccaacctaatcgaccagaacgtattgtggaaagttttaaagctctttgcaccttaagaaatgaacctgctgaattgattaaaaaacttacatgggaaaatgcttgccagattttttct

SEQ ID NO: 40 - protein, 262 aa, H. influenzae
MHFFDTHTHLNYLQQFTGEPLSQLIDNAKQADVQKILVVAVKEADFKTIQNMTALFPDNLCYGLGLHPLYIQEHAENDLILLEQALKNRDTNCTAVAEIGLERAIPDLLTDELWAKQCHFFESQLYLAKQFNLPVNIHSRKTHDQIFTFLKRIPLSKLGVVHGFSGSYDQAKRFVDLGYKIGVGGTITYERANKTRQAIAKLPLDALVLETDSPDMPVFGFQGQPNRPERIVESFKALCTLRNEPAELIKKLTWENACQIFS

SEQ ID NO: 41 - DNA, 264 bp, H. influenzae
  CDS (1)...(264): HI-0082
atgggattaaccctaaaagaacacgctgaagtctgtatggcattagctgaaagttcagcctctgctggtctctgttatatgatgagtaacgttgcagtgaattgtttaaatctattcggcagctatcaactcaaacaaaaaatcttttctgacattgttcaaaataaaacctttgccgcactcgcctatagtgaattaggcacgggtactcacttctattcgagtttttacatcaacatggcttggaatttggtcaagattatg

SEQ ID NO: 42 - protein, 88 aa, H. influenzae
MGLTLKEHAEVCMALAESSASAGLCYMMSNVAVNCLNLFGSYQLKQKIFSDIVQNKTFAALAYSELGTGTHFYSSFYINMAWNLVKIM

SEQ ID NO: 43 - DNA, 213 bp, H. influenzae
  CDS (1)...(213): HI-0083
gtgggtgttggtttgcacggtgatcatgtaggcggtgaattaaattcagccaatgcattcaccgaaaccttgttcaaaatggattacaacaatccagaacataaagaaatgatggatttggaaggattaaaacgttggattgcgcgcagaaaaagccttaaattaccttccaccagagcaaatatcaaaatttcagacaaaaaattgccccat

SEQ ID NO: 44 - protein, 71 aa, H. influenzae
VGVGLHGDHVGGELNSANAFTETLFKMDYNNPEHKEMMDLEGLKRWIARRKSLKLPSTRANIKISDKKLPH

SEQ ID NO: 45 - DNA, 711 bp, H. influenzae
  CDS (1)...(711): HI-0090
atgaatatccagcataatctcaacctaattcagcaaaaaattgaaacggcttgtaaagaagaaaatcgcaatcaaaataccgtaaaattacttgccgtatctaagaccaaacctatttctgctattctttcggcttatcaagcgggacaaacggcttttggggaaaattacgtgcaagagggggtagaaaagatccaatattttgaatcgcaaggaattaaccttgaatggcattttatcggcccattacaatcgaataaaacccgtctcgttgcagaacattttgattggatgcaaacgcttgatcgagcaaaaattgcagatcgtttaaatgaacaacgaccgaccaacaaggctccattgaatgtattgattcagattaatatcagcgatgaagaaagtaaatcaggtattcaacctgaggaaatgctaacactagcaaaacacatcgaaaatttaccgcacttatgcctacgtggcttaatggcaatacccgcaccaaccgacaacatcgcagaacaagaaaatgcatttagaaagatgttggagctgtttgaacaacttaaacaagtcttacccaatcaacaaattgatacactttctatgggaatgacggatgatatgccgagcgcaataaaatgcggttctacaatggtacgcattggcactgcgatttttggtgcgagaaattattcaacatcacaaaataaa

SEQ ID NO: 46 - protein, 237 aa, H. influenzae
MNIQHNLNLIQQKIETACKEENRNQNTVKLLAVSKTKPISAILSAYQAGQTAFGENYVQEGVEKIQYFESQGINLEWHFIGPLQSNKTRLVAEHFDWMQTLDRAKIADRLNEQRPTNKAPLNVLIQINISDEESKSGIQPEEMLTLAKHIENLPHLCLRGLMAIPAPTDNIAEQENAFRKMLELFEQLKQVLPNQQIDTLSMGMTDDMPSAIKCGSTMVRIGTAIFGARNYSTSQNK

SEQ ID NO: 47 - DNA, 1134 bp, H. influenzae
  CDS (1)...(1134): HI-0091
atgaaatttgtgattgcacctgattcttttaaagaaagtttgaccgcacttgaagtcgctaccgccattgaaacaggcttcaaacgtgtattccccgatgcggactatgtaaaattgccaatggcagatgggggcgaaggtacagttcaatcccttgtggatgccacgcaaggcaagctcattgaatgtgaagtcaccgcgcctttgggtgacaaagtgaaaagtttcttcggtttatcgggagatggaaaaaccgcgattatcgaaatggcagcagcttctggtttacatcttgttccgcctgaaaaacgcaatcccttgcttaccaccagttacggcacaggggagttgattaaactggcattagatttaggcgtagagagtttcattcttggcattggcggtagcgcaaccaacgatggtggtgtaggaatgttgcaagcactagggatgcaatgtttagattcgcaagataagcctatcggttttggtggagcagagctagcaaatattgtgaagattgacgttcaacaattagatccacgtttgcaacaagttcacattgaagtagcgtgcgatgtgaataatccgctctgtggtgaatgtggtgcatctgccatttttggaccacaaaaaggcgcaacgcctgaaatggtgaaacaacttgatgctgcactttcgcattttgctgaaattgccgaacgcgattgcggtaaacaaattcgtgatcaggctggcgcaggtgctgctggtggtatgggtggtggcttgctattattaccaagcgtacaactgaaagcaggcatacaaattgtgttagaccggttacatttaattgactatgtaaaagatgctgatgtagtgattacgggtgaagggcgaatagatgcacaaagtattatgggtaaaacgccaattggtgtggcacgtacggcaaaacagttcaataagccagtgatcgccatcgcagggtgtttgcgtgaagattatgatgtggtgtttgatcacggtattgatgcggtgttccctattattcaccaacttggcgatttatcggatattctcaaacaaggcgaacaaaatttaatttctaccgctcaaaatgtggcgagagtactggcatttaaatttcat

SEQ ID NO: 48 - protein, 378 aa, H. influenzae
MKFVIAPDSFKESLTALEVATAIETGFKRVFPDADYVKLPMADGGEGTVQSLVDATQGKLIECEVTAPLGDKVKSFFGLSGDGKTAIIEMAAASGLHLVPPEKRNPLLTTSYGTGELIKLALDLGVESFILGIGGSATNDGGVGMLQALGMQCLDSQDKPIGFGGAELANIVKIDVQQLDPRLQQVHIEVACDVNNPLCGECGASAIFGPQKGATPEMVKQLDAALSHFAEIAERDCGKQIRDQAGAGAAGGMGGGLLLLPSVQLKAGIQIVLDRLHLIDYVKDADVVITGEGRIDAQSIMGKTPIGVARTAKQFNKPVIAIAGCLREDYDVVFDHGIDAVFPIIHQLGDLSDILKQGEQNLISTAQNVARVLAFKFH

SEQ ID NO: 49 - DNA, 1104 bp, H. influenzae
  CDS (1)...(1104): HI-0093
atgcaacttgacaaatacactgccaaaaaaattgttaaacgtgcaatgaagatcattcatcactctgtaaatgtaatggatcacgacggtgtgatcattgcgtcgggaaattcaacgcgtttgaatcaacggcacacaggagcggtgttggcgttgcgggaaaatcgtgtggtagagattgatcaagcgttggcacaaaaatggaattttgaagcacaaccagggattaatttaccgattcattatttaggcaaaaatattggagtggtggggatttctggtgagccaactcaggtgaaacaatatgccgaactagtgaaaatgacggcagaacttattgtggaacaacaggctttactcgaacaagaaagctggcatcgtcgttacaaagaagaatttattttacaattattacattgcaatttgaattggaaagagatggaacagcaggcgaaatttttttcttttgatttaaataaatctcgtgtggttgtattgattaagttacttaatccagccctggataatttacagaatctgatcaattatttggaacagtctgaatttgcacaggatgtggcaattttgtcccttgatcaggttgtggtcttaaagacttggcaaaactccaccgtactttctgctcaaatgaaaacgcttttacctgcagattattcaaaacaagattataaaattgctgtaggcgcttgtctaaatctaccgctttttgagcagcttcccttgtcttttcaaagtgcgcaaagcacgctttcttatggattaaaacatcatccacgcaaaggtatttatgtgtttgatgagcatcgattgcctgtgttacttgctggattatcccattcttggcaagggaacgaattgataaaacccctttctccccttttctcagaagaaaatgcgatactttataaaacccttcaacaatattttttatcaaattgtgatctttatctcactgcggaaaaattatttgttcacccgaatactttgcgttatcgattgaacaaaatagagcaaataactggcttatttttcaataagatagatgataaattaacgctctatctcggaacgttgttagaacat

SEQ ID NO: 50 - protein, 368 aa, H. influenzae
MQLDKYTAKKIVKRAMKIIHHSVNVMDHDGVIIASGNSTRLNQRHTGAVLALRENRVVEIDQALAQKWNFEAQPGINLPIHYLGKNIGVVGISGEPTQVKQYAELVKMTAELIVEQQALLEQESWHRRYKEEFILQLLHCNLNWKEMEQQAKFFSFDLNKSRVVVLIKLLNPALDNLQNLINYLEQSEFAQDVAILSLDQVVVLKTWQNSTVLSAQMKTLLPADYSKQDYKIAVGACLNLPLFEQLPLSFQSAQSTLSYGLKHHPRKGIYVFDEHRLPVLLAGLSHSWQGNELIKPLSPLFSEENAILYKTLQQYFLSNCDLYLTAEKLFVHPNTLRYRLNKIEQITGLFFNKIDDKLTLYLGTLLEH

SEQ ID NO: 51 - DNA, 318 bp, H. influenzae
  CDS (1)...(318): HI-0094
atgagtgaattacttattaatgactatacaagaaaaggttttgttgatggtctctgtcttcgcctgcccacaatttgtatccgtccaggaaaaccaaataaagcgacttcttcctttgtaagtagcattattcgagaacctttacatggtgaaacttcaatctgcccagttgctgaaaaaatggcgttcagctttatcaaatttctaggtaagaaaaaggaagaatgggcattagccattacgggctatgtcgtgagtattcctattgtcttaccgattttaattattttcatcaaagctattcttgatttggggaaa

SEQ ID NO: 52 - protein, 106 aa, H. influenzae
MSELLINDYTRKGFVDGLCLRLPTICIRPGKPNKATSSFVSSIIREPLHGETSICPVAEKMAFSFIKFLGKKKEEWALAITGYVVSIPIVLPILIIFIKAILDLGK

SEQ ID NO: 53 - DNA, 753 bp, H. influenzae
  CDS (1)...(753): HI-0095
atggcaaaagatgaagtagggcataattttcttgcacgtttgggtaaaacgcgtttgcgtccaggcggtaaaaaagcgacagattggttaattgctaatggcggttttagccaagataaaaaagtgttggaggttgcctgtaatatggggacgactgcaattggattggcgaaacaatttggttgtcatattgaaggtgttgatttagatgaaaatgcgttagcaaaagcacaagcaaatattgaagcaaatggcttgcaggaaaaaattcatgtacagcgtgcgaatgcgatgaagttgcctttcgaggatgaaagttttgatattgtcatcaatgaagcgatgctcacaatgttacccgtggaagcgaagaaaaaagccattgcagaatattttcgagtgttaaaacccaatggtttattgcttactcacgatgttatgctggtggggaatgatcatcaaactattctagaaaatatgcgcaaagcgattaacgtgactgtcacgccattaacgaaagatggatggaaaggcatattccaagaaagtggttttagaaatgttgatactttctctggtgagatgacattactttccccaaaaggaatgatttatgatgaaggaattttcggtacgttaaaaatcatccgtaatgcgatgaaagcggaaaatcgtgagcaatttaaaagaatgttcaaaacctttaatgatcctgaacataaattacattttattgctgtatgtagccaaaaa

SEQ ID NO: 54 - protein, 251 aa, H. influenzae
MAKDEVGHNFLARLGKTRLRPGGKKATDWLIANGGFSQDKKVLEVACNMGTTAIGLAKQFGCHIEGVDLDENALAKAQANIEANGLQEKIHVQRANAMKLPFEDESFDIVINEAMLTMLPVEAKKKAIAEYFRVLKPNGLLLTHDVMLVGNDHQTILENMRKAINVTVTPLTKDGWKGIFQESGFRNVDTFSGEMTLLSPKGMIYDEGIFGTLKIIRNAMKAENREQFKRMFKTFNDPEHKLHFIAVCSQK

SEQ ID NO: 55 - DNA, 573 bp, H. influenzae
  CDS (1)...(573): HI-0096
gtggatttagcggatagtcaaattacccaaggtaatgaaattattcaatcaatgggcttaacaaacgttcgtttgcttgaatattttatttatcaagttggttcatttactattcaatctttaacacagcatattgaggaaaataaagaatttgccaatattactgaaaatgaactgtattctgcggtgctatctttagttattttaggttatgtctatgtttatcttacgacctatccaatttattcctttgaagataacaaaacttacataccgaaaagctttactcaatatgtaaaaacattggtagaaggcgcaaatcagtatattggtgctggaaatatgtataacggcgacgtagaagatttgaataaactgcatctttatataatgtctcaaatggaaaaaccgacaactaaagctgaattgaaatcagcattacaaggctatttaattcaaaatgaatatcaagatatgaataacaatgataaattgattgatgaaacttatgattgcactgagttgtttaatgcgctttttgatgtattaacaagattaggtatatccagcctg

SEQ ID NO: 56 - protein, 191 aa, H. influenzae
VDLADSQITQGNEIIQSMGLTNVRLLEYFIYQVGSFTIQSLTQHIEENKEFANITENELYSAVLSLVILGYVYVYLTTYPIYSFEDNKTYIPKSFTQYVKTLVEGANQYIGAGNMYNGDVEDLNKLHLYIMSQMEKPTTKAELKSALQGYLIQNEYQDMNNNDKLIDETYDCTELFNALFDVLTRLGISSL

SEQ ID NO: 57 - DNA, 393 bp, H. influenzae
  CDS (1)...(393): HI-0100
atgtcaggcgattttacgttggtctgcattactgcggctagtcgtcatcattggggaacggaagtggatatttttgatcctgatcttttgccacgaggtcaatctttacaactggagccttgggaatatgaaaaaggcggctacttctttgaattgagtgaatttcttgccgaaaatttaccgcactttgattttgctttgccttttatgaatatgcagtccaataagaaagtggggcgggagccttggcatatcagttatttgccgttagctgaattggcaagccagcaattttctccagagattttgccacaggcgtggaaaggggaaaatattttaggcgcagactgtttaatatcacatcttgaacaaattttttccgaatatattgtt

SEQ ID NO: 58 - protein, 131 aa, H. influenzae
MSGDFTLVCITAASRHHWGTEVDIFDPDLLPRGQSLQLEPWEYEKGGYFFELSEFLAENLPHFDFALPFMNMQSNKKVGREPWHISYLPLAELASQQFSPEILPQAWKGENILGADCLISHLEQIFSEYIV

SEQ ID NO: 59 - DNA, 438 bp, H. influenzae
  CDS (1)...(438): HI-0101
atgaaattaaccccagaaatgcttacaggaaagtcccgtgagcatttagtcaatttacccacaactcattcatcaaatcattttttgcaaacgcaagctgtgcaagcatttcaagctttgcaacaaagtgcggcaaaaaatggctttaatttacagcctgcgagcagttttcgtgattttgaacgccaacaacttatttggaacagtaaatttaagggcgagcgaaaagtacacgatgatgcaggaaaggcattagatttgaatcaattagatgactggcaaaaatgtcaggcgattttacgttggtctgcattactgcggctagtcgtcatcattggggaacggaagtggatatttttgatcctgatcttttgccacgaggtcaatctttacaactggagccttgggaatatgaaaaaggcggctacttctttgaat

SEQ ID NO: 60 - protein, 146 aa, H. influenzae
MKLTPEMLTGKSREHLVNLPTTHSSNHFLQTQAVQAFQALQQSAAKNGFNLQPASSFRDFERQQLIWNSKFKGERKVHDDAGKALDLNQLDDWQKCQAILRWSALLRLVVIIGERKWIFLILIFCHEVNLYNWSLGNMKKAATSLN

SEQ ID NO: 61 - DNA, 1893 bp, H. influenzae
  CDS (1)...(1893): HI-0104
atgaaggaaaaaattatgtcacaaaatcaagaaactcgcggattccaatctgaagtcaaacaactactccaattaatgattcattctttgtactctaacaaagaaattttcttacgtgaattgatttctaatgcttctgatgcggcagataaattacgctttaaagcactttccaaccctgctttatatgaaggcgatggcgacttgcgtgtgcgtgttagctttgatgcggataaaggcactatcacaattagcgataacggcattggcatgacccgtgagcaagtcatcgatcatttgggtacgattgcaaaatcaggaacaaaagaatttttaaccgcacttggtcaagatcaagcaaaaaatagccagcttattgggcagtttggtgtgggtttttattctgcttttattgtggcagataaagtgactgtaaaaaccagagcagctggtgaagaggcggataaagccgtactttgggaatctgcgggtgaaggcgaatattctgtggcggatattgagaaaaaatcccgtggtacagatgtgattttgcatttacgtgaagatgaaaaagaatttttaaatgaatggcgtttgcgtgaaattattggtaaatattctgaccatattggcttgccagtggaaatgctgacgaaagaatacgatgatgaaggcaaagagtgcggcgaaaaatgggaaaaaatcaataaatctgatgccctttggacgcgttctaaaaatgatgtgtctgatgaggaatacaaagcgttttacaaacacttaagccatgattttgttgatcctgtgacttgggcgcataacaaagttgagggaaatcaagcatatactagtttgctttatgtaccagctaaagcaccttgggatttatttaatcgagaacataaacatggcttaaaactttatgttcaacgtgtatttattatggatgatgctgagcaatttataccgaattatctacgttttatgcgtggtttaatcgatagcaatgacttgccattaaatgtatctcgtgaaattttacaggataacaaaattacggctgcactacgcaaagcattaactaagcgttcattacaaatgctagaaaaattagcaaaagatgatgcagaaaaatatcttcaattctggaaagagtttggtttagtcttaaaagaaggccctgcagaagattttgctaacaaagaaactgtcgcgaagttattgcgttttgcttcgacacataatgatggtagcgagcaaactgtctctttagaagattacatcttgcgtatgaaagaggggcaaaaagctatctattacattacagcggatagttatgttgcagcgaaaaatagcccgcacttggaattattcaataaaaaaggcattgaggttttgttgctttccgatcgcattgatgaatggatgttaagctatttaactgaattcgatggtaaacaattacaaagtattacaaaagcagatttggatttaggcgatttagcggataaagaatctgaaacacaaaaacaacaagatgaagcctttggtagttttattgagcgtgtgaaaaacttgcttggcgaacgtgtgaaaacggtgcgtttaactcacaatttaacggatacaccagcggtggtttctacggataacgatcaaatgacgacccaaatggcgaaattgtttgcagccgcagggcaacctgtaccagaagtaaaatacacatttgaactgaatccagaacaccatttagtgaaaaaagtggctgatattgcagatgaaactgaatttgctgattgggtggaattattacttgaacaagctatgttagcagagcgcggatctttggaaaatccagctgcgtttattaaacgtatcaataagttgttaggt

SEQ ID NO: 62 - protein, 631 aa, H. influenzae
MKEKIMSQNQETRGFQSEVKQLLQLMIHSLYSNKEIFLRELISNASDAADKLRFKALSNPALYEGDGDLRVRVSFDADKGTITISDNGIGMTREQVIDHLGTIAKSGTKEFLTALGQDQAKNSQLIGQFGVGFYSAFIVADKVTVKTRAAGEEADKAVLWESAGEGEYSVADIEKKSRGTDVILHLREDEKEFLNEWRLREIIGKYSDHIGLPVEMLTKEYDDEGKECGEKWEKINKSDALWTRSKNDVSDEEYKAFYKHLSHDFVDPVTWAHNKVEGNQAYTSLLYVPAKAPWDLFNREHKHGLKLYVQRVFIMDDAEQFIPNYLRFMRGLIDSNDLPLNVSREILQDNKITAALRKALTKRSLQMLEKLAKDDAEKYLQFWKEFGLVLKEGPAEDFANKETVAKLLRFASTHNDGSEQTVSLEDYILRMKEGQKAIYYITADSYVAAKNSPHLELFNKKGIEVLLLSDRIDEWMLSYLTEFDGKQLQSITKADLDLGDLADKESETQKQQDEAFGSFIERVKNLLGERVKTVRLTHNLTDTPAVVSTDNDQMTTQMAKLFAAAGQPVPEVKYTFELNPEHHLVKKVADIADETEFADWVELLLEQAMLAERGSLENPAAFIKRINKLLG

SEQ ID NO: 63 - DNA, 1314 bp, H. influenzae
  CDS (1)...(1314): HI-0125
atgccaactttagaaaaaacatttgagctcaaacaacgtggttcaactgttcgtcaagaaattatcgcgggtttaaccactttcttagcaatggtttattctgtcatcgtggtacccaatatgcttggtgctgcaggtttccctgcggaatctgtctttattgccacctgtttagttgcaggattaggctctatcttaatcggcttatgggcaaatgcaccaatggcaattggttgtgcaatttctcttactgcttttacagcatttagcttagtgattggtcaaaaggtcgccattcccgttgcgctaggcgcagtattcttaatgggggttgtgtttaccttaatttctaccacaggtatccgtgcatggatcttgcgtaatttaccatccaatatcgctcatggagcaggcattggtatcggactctttttacttttaattgccgcaaacggtgttggtttagtcgtcagcaaccaagctggcttaccagtaaaattaggcgatttcacctcattcccagtcatgatgtccttaattggcttagcgttaattattggccttgaaaaaatgaaaattaaaggcggcattttatgggtaattatcgctattactatcgtaggtttaatttttgatccaaatgtaaaatttggcggagagattttcaaaatgccaacctttggtgaaaattctcttttcttacaattagactttatgggcgcactacaacccgcaatcttgcctgtggtatttgctttggtgatgactgccgtatttgatgcaaccggtacaattcgtgcagttgcaggtcaagcagacttgcttgataaagatggtcaaattatcaatggcggtaaagccttaacttcagattcaatcagtagcttattctcaggcttattcggtacagcaccagctgccgtttatattgagtcagcagcagggacagcagcaggcggtaaaacaggtattactgctatcgttgttggtgtgttattcctattaatgcttttcttccaaccattagctttcttagtgccaggctacgcaactgctccagcattaatgtatgttggtttattaatgttaagcaatgtaagcaaattagatttcgatgatttcgttggcgcgatgagcggattaatctgcgcagtgtttatcgtacttaccgcgaacatcgtaacaggcattatgttaggttttgcggcattagtaattggaagaatcgtgagtggcgatattaaacgcttaaatgtaggtactgtgattattgcaatcgtacttgttgcattctatgcaggcggttgggcgatt

SEQ ID NO: 64 - protein, 438 aa, H. influenzae
MPTLEKTFELKQRGSTVRQEIIAGLTTFLAMVYSVIVVPNMLGAAGFPAESVFIATCLVAGLGSILIGLWANAPMAIGCAISLTAFTAFSLVIGQKVAIPVALGAVFLMGVVFTLISTTGIRAWILRNLPSNIAHGAGIGIGLFLLLIAANGVGLVVSNQAGLPVKLGDFTSFPVMMSLIGLALIIGLEKMKIKGGILWVIIAITIVGLIFDPNVKFGGEIFKMPTFGENSLFLQLDFMGALQPAILPVVFALVMTAVFDATGTIRAVAGQADLLDKDGQIINGGKALTSDSISSLFSGLFGTAPAAVYIESAAGTAAGGKTGITAIVVGVLFLLMLFFQPLAFLVPGYATAPALMYVGLLMLSNVSKLDFDDFVGAMSGLICAVFIVLTANIVTGIMLGFAALVIGRIVSGDIKRLNVGTVIIAIVLVAFYAGGWAI

SEQ ID NO: 65 - DNA, 984 bp, H. influenzae
  CDS (1)...(984): HI-0126
atgagtaataatgatttcttagtattaaaaaatatcacaaaagcatttggtaaagcggtcgtcattgataatttagatttaacgatcaaacgtggcacaatggtaacattgttagggccatcaggctgtggtaaaaccaccgtattacgtttagtggcaggattagaaaatccaacatcaggtcaaatatttattgatggcgaagatgtaacaaaatcctctattcagaatcgagatatttgtattgttttccaatcttacgcgcttttcccgcatatgagcattggcgataacgtgggctacggcttaaaaatgcaaggcattggcaaagaagaacgcgctcagcgtgtaaaagaggctttagaattagtggatttagcgggctttgaagatcgtttcgtggatcaaatttctggtgggcaacaacaacgtgtcgcattggctcgtgctttggtattgaaaccaaaagtcctattgtttgatgaaccattaagtaatttagatgcaaacttacgtcgttctatgcgtgaaaaaatccgtgaattgcaacaacgtttaggcattacttcgctttatgtgacacacgatcaaacagaggcatttgcggtatctgatgaagtgattgtgatgaataaaggtaaaattatgcaaaaagcgccggcgaaagagctttatctccgaccaaattctctgtttttggctaactttatgggcgaatccagtattttcgatggaaaattagaaaatggcgtggcggatattaatggttactctgtgcctttaaaagatgctgcacagtttaatttacccgatggcgaatgtttagtgggtattcgcccagaagcaatttatcttgctgcagagggtagcgatgcacaacgttgcgagattaaaagtgcggtttatatggggcaaccattggggaagtggttgcaaactgggcggggcaaagatttactggtaaattgtaaaccagagggattt

SEQ ID NO: 66 - protein, 328 aa, H. influenzae
MSNNDFLVLKNITKAFGKAVVIDNLDLTIKRGTMVTLLGPSGCGKTTVLRLVAGLENPTSGQIFIDGEDVTKSSIQNRDICIVFQSYALFPHMSIGDNVGYGLKMQGIGKEERAQRVKEALELVDLAGFEDRFVDQISGGQQQRVALARALVLKPKVLLFDEPLSNLDANLRRSMREKIRELQQRLGITSLYVTHDQTEAFAVSDEVIVMNKGKIMQKAPAKELYLRPNSLFLANFMGESSIFDGKLENGVADINGYSVPLKDAAQFNLPDGECLVGIRPEAIYLAAEGSDAQRCEIKSAVYMGQPLGKWLQTGRGKDLLVNCKPEGF

SEQ ID NO: 67 - DNA, 1038 bp, H. influenzae
  CDS (1)...(1038): HI-0131
atgaaattcaacaaaatttctctttctgtttctaccgcacttttagctgctggcttggctgtttctggttctgctaacgctaaaggtcgtttagttgtatattgtagtgcaaccaatattttgtgcgaaaccaccacgaaagcatttggcgaaaaatatgatgtgaaaacatcctttattcgtaatggttcaggcagtacttttgctaaagttgaagctgaaaaaaataaccctcaagcggatgtttggttcggcggtacttttgaccctcaagctcaagcggcagaattagggttaattgagccttataaatccaaacatattgatgaaattgtagaacgtttccgtgaaccagcgaaaacgaaaggccattatgtttcctcaatttatatggggatcttaggtttcggtgtgaatactgaacgtttggcaaaattaggtattaaagaagtgccaaaatgctggaaagacttaaccgatccacgcttaaaaggtgaagttcaaattgcagaccctcaaagtgcgggtactgcttacactgcattggcaactttcgttcaattatggggcgaaaaagaggcattcgatttcctaaaagagttacatcctaatgtttctcaatataccaaatcgggtatcacgccatcacgtaactctgcgcgtggcgaagcgacaattggggtgggtttcttacacgattatgctttagaaaaacgcaatggtgcgccattagaattagttgtgccgtgcgaaggaacgggctatgaattaggtggcgtgagtatcttaaaaggtgcgcgtaatattgataatgcaaaattattcgtcgattgggctttatcaaaagaaggtcaagaattagcttggaaacaaggggattctttacaaatcttaactaacacgaccgcagaacaatcgccaactgcatttgatccaaataaactcaaattaatcaattatgactttgaaaaatacggtgcaacagaacaacgcaaagccttaattgaaaaatgggttcaagaagttaaattggcgaaa

SEQ ID NO: 68 - protein, 346 aa, H. influenzae
MKFNKISLSVSTALLAAGLAVSGSANAKGRLVVYCSATNILCETTTKAFGEKYDVKTSFIRNGSGSTFAKVEAEKNNPQADVWFGGTFDPQAQAAELGLIEPYKSKHIDEIVERFREPAKTKGHYVSSIYMGILGFGVNTERLAKLGIKEVPKCWKDLTDPRLKGEVQIADPQSAGTAYTALATFVQLWGEKEAFDFLKELHPNVSQYTKSGITPSRNSARGEATIGVGFLHDYALEKRNGAPLELVVPCEGTGYELGGVSILKGARNIDNAKLFVDWALSKEGQELAWKQGDSLQILTNTTAEQSPTAFDPNKLKLINYDFEKYGATEQRKALIEKWVQEVKLAK

SEQ ID NO: 69 - DNA, 1512 bp, H. influenzae
  CDS (1)...(1512): HI-0136
atggcaactccagtcgtcgcccttgttggtcgcccaaatgtgggaaaatccacattatttaatcgccttactcgtacgagagatgcgttagtcgctgattttcccggtttaactcgtgatagaaaatatggtcacgcacatattgctggctatgattttattgttattgatactggcggtattgatggaacggaagagggtgtagaagaaaaaatggcggagcaatctttgcttgctattgatgaagcggatattgttcttttccttgtggatgctagagcgggtttaacggcagctgatattggtattgctaattatttacgccaacgtcaaaacaaaattactgtggtggtggcaaataaaactgatggtattgatgcggattcccattgtgctgaattttatcagttaggtttaggggaaattgagcaaatcgcagcctcacaaggccgtggtgttactcaattaatggaacaagtacttgcgccttttgcggaaaaaatggaaaacgccgatgaaaatgaccgcacttctgaggaagaacaagacgaatgggaacaagaattcgattttgattcagaagaagatacggcattaattgatgatgcgttagacgaagaacttgaagaagaacaagataaaaatattaagattgccattgttggtcgtccaaatgtgggtaaatctactttaaccaatcgtattttaggcgaagatcgtgtcgtggtttttgatatgccaggtacgacacgtgacagtatttatattccaatggagcgtgatgggcagcaatataccttgattgatacggctggtgtgcgtaagcgtggtaaagtgcatttggcagtggaaaaattctctgtgattaaaaccttacaagcgattcaagatgctaatgttgtattgctgactattgatgcgagagagaacatttctgatcaggatttatctttgctcggctttattttaaatgcagggcgttctttggtgatcgtcgtgaataaatgggatggtttagatcaagatgtgaaagatcgtgtgaaatctgaacttgatcgtcgtttagattttattgattttgctcgggtgcattttatttcagccttgcacggaagtggtgtaggtaatctttttgattcgattaaagaagcctatgcttgtgccactcaaaaaatgacgacttcgcttttaactcgtattttgcaaatggcaacggatgagcaccaaccgccgatgattggcggtcgtcgcattaaattaaaatatgctcacccaggtggttataatccgccgattatagtggttcacggtaaccaaatggataaactaccagattcttataaacgttatttatctaattattatcgtaagagcttaaaaattattggttcaccgattcgtctgcttttccaagaaggctcaaacccatttgcaggacgcaaaaataaactcactccgaaccaattgcgtaaacgtaaacgtttgatgaaatttattaagaaagcgaaacgt

SEQ ID NO: 70 - protein, 504 aa, H. influenzae
MATPVVALVGRPNVGKSTLFNRLTRTRDALVADFPGLTRDRKYGHAHIAGYDFIVIDTGGIDGTEEGVEEKMAEQSLLAIDEADIVLFLVDARAGLTAADIGIANYLRQRQNKITVVVANKTDGIDADSHCAEFYQLGLGEIEQIAASQGRGVTQLMEQVLAPFAEKMENADENDRTSEEEQDEWEQEFDFDSEEDTALIDDALDEELEEEQDKNIKIAIVGRPNVGKSTLTNRILGEDRVVVFDMPGTTRDSIYIPMERDGQQYTLIDTAGVRKRGKVHLAVEKFSVIKTLQAIQDANVVLLTIDARENISDQDLSLLGFILNAGRSLVIVVNKWDGLDQDVKDRVKSELDRRLDFIDFARVHFISALHGSGVGNLFDSIKEAYACATQKMTTSLLTRILQMATDEHQPPMIGGRRIKLKYAHPGGYNPPIIVVHGNQMDKLPDSYKRYLSNYYRKSLKIIGSPIRLLFQEGSNPFAGRKNKLTPNQLRKRKRLMKFIKKAKR

SEQ ID NO: 71 - DNA, 462 bp, H. influenzae
  CDS (1)...(462): HI-0138
atgcckaaacagattgaaatttttactgatggatcttgcttaggtaatccaggggcgggcggaattggtgccgtattgcgttataaacaacatgaaaaaacactctccaaaggctatttccaaaccaccaataatcgaatggaattacgcgctgtcattgaagcattaaatacattaaaagaaccttgcttgatcacgctttatagtgatagccaatatatgaaaaatggcataaccaaatggatctttaactggaaaaaaaataattggaaagcaagttctggaaagcctgtaaaaaaccaagatttatggatagccttagatgaatccatccaacgtcataaaattaattggcaatgggtaaaaggccatgctggacacagagaaaatgaaatttgcgatgaattagcaaaaaaaggggcagaaaatccgacattggaagatatggggtacatagaagaa

SEQ ID NO: 72 - protein, 154 aa, H. influenzae
  VARIANT (2)...(2): Xaa = Any amino acid
MXKQIEIFTDGSCLGNPGAGGIGAVLRYKQHEKTLSKGYFQTTNNRMELRAVIEALNTLKEPCLITLYSDSQYMKNGITKWIFNWKKNNWKASSGKPVKNQDLWIALDESIQRHKINWQWVKGHAGHRENEICDELAKKGAENPTLEDMGYIEE

SEQ ID NO: 73 - DNA, 705 bp, H. influenzae
  CDS (1)...(705): HI-0152
atgacaacctacatcgcctacggcaatataaatcaacctttttctttggaatccttacccgatgaactgattccagaaaatctatatcaaattgaaacggatagctcccgtgtttttcagcgtcatcagtgtcgtcggcttgcgcatttattacttttccaacttctaaaaatagcaggaaaatccaccgcacttttatctcaaattcatcgtactgaaagtggcagaccttattttcttgatgagcgaatagattttaatattagccattctggtgattgggtggcggtaatattagatattagaaatgaagaaaaaagtgcggtgggaattgatattgaatttcctaaaataagaaattttacggcgttgatggaacatattgcaccaaaagaagaaattgattggtttcatcatcagcaggattctttgaacgctttttatcgttgttggtgtttgcgagaggctgtattgaaatctcaaggatttgggatcgtaaaattatccaatgttcgtcattttcctgaacaacaaaaaattttttcagattattgtccgcaggggcagttgtggtttactgatgaactccctatttatttagccgcttttgtcaatcatcaagaaaaattaccgcacttttatgaatggaatagagaaagtttacagataaaagaacttgaaaaatatgttctttatgaagtgaat

SEQ ID NO: 74 - protein, 235 aa, H. influenzae
MTTYIAYGNINQPFSLESLPDELIPENLYQIETDSSRVFQRHQCRRLAHLLLFQLLKIAGKSTALLSQIHRTESGRPYFLDERIDFNISHSGDWVAVILDIRNEEKSAVGIDIEFPKIRNFTALMEHIAPKEEIDWFHHQQDSLNAFYRCWCLREAVLKSQGFGIVKLSNVRHFPEQQKIFSDYCPQGQLWFTDELPIYLAAFVNHQEKLPHFYEWNRESLQIKELEKYVLYEVN

SEQ ID NO: 75 - DNA, 870 bp, H. influenzae
  CDS (1)...(870): HI-0160
atgatgacttcaaattcttattggcaacgcttaaaagtggcatttcaatatgtcatgccacaaatttatttaactcaaattgctggctggtttgctaaacaaaaatggggcaaaataacacattttgtaattaaagcttttgcgaaaaaatacaacattgatatgagcattgcgcaaaaagaacaattttctgattacgcaagttttaatgaattttttattcgtccgttaaaagaaaacgcacgtccaattaatcaaaatccaaccgcactttgtttaccagcagacggtcgcattagtgagtgcggtcatattgacgataatcttttattgcaagctaaggggcattttttcagcctagaagacttattggcagaagataaagaattagtggaaacctttaaaaatggggaatttgtaactacttatctttctcctcgtgattatcaccgagtgcatatgccatgcgatgctacgctacgcaaaatgatttatgtgccgggtgatttattctctgtgaacccatttttagcccaacatgtaccaaatttatttgcacgtaatgaacgtgtgatttgtgtatttgatactgaatttggcacaatggtacaaattttagtgggtgcaaccatcactgcaagtattggcacaacttgggcaggcgtaattaatcctccacgccacaacgaagtgaaaacttggacttatgaaggcgaaagtgcggtcaaattattgaaaggtcaagaaatggggtggttccaacttggttcgacagtaattaatttattccaagcaaatcaagtgcgtttagctgatcatttaagcgttaatgaacctgttcgcatgggcgaaatcttggcatataaaaaa

SEQ ID NO: 76 - protein, 290 aa, H. influenzae
MMTSNSYWQRLKVAFQYVMPQIYLTQIAGWFAKQKWGKITHFVIKAFAKKYNIDMSIAQKEQFSDYASFNEFFIRPLKENARPINQNPTALCLPADGRISECGHIDDNLLLQAKGHFFSLEDLLAEDKELVETFKNGEFVTTYLSPRDYHRVHMPCDATLRKMIYVPGDLFSVNPFLAQHVPNLFARNERVICVFDTEFGTMVQILVGATITASIGTTWAGVINPPRHNEVKTWTYEGESAVKLLKGQEMGWFQLGSTVINLFQANQVRLADHLSVNEPVRMGEILAYKK

SEQ ID NO: 77 - DNA, 1368 bp, H. influenzae
  CDS (1)...(1368): HI-0161
atgactaaacattatgattatattgctattggcggtggcagtggcggtattgcgtctctaaatcgagcagcaagctatggaaaaaaatgtgcaatcattgaagcaaaacatcttggcggaacttgtgtaaatgtcggttgtgtacctaaaaaagtgatgttttatggtgcgcatattgcagaagcaatcaacaattatgcgccagattatggttttgatgttgaagtgaaaaaatttgatttttcaaaactgattgaaagtcgccaagcctatattagtcgtatccatacatcttataataatgtattagcgaaaaataatattgatgtaattaatggtttcggaaaatttgtcgatgcccataccattgaagtaacacttgctgatggtacaaaagaacaagtaaccgcagatcatattttaatcgcaactggtggtcgtccatatcgtccaaatattaaaggacaagaatacggcattgattcagatggtttctttgcattaaccgaattaccaaaacgtgctgctgttattggtgcaggctatattgctgttgaactttctggcgtattaaatagcttaggcgtggaaacacatttattagtgcgtcgccatgcgccaatgcgtaatcaggatccattaatcgtagaaacattagtggaagtgcttgcgcaagatggaattcaattacataccaattctaccccatctgaaattgtaaaaaatgcagatggttcacttactgtaagatgtgatggtcaatctgatgttaccgtagattgcgttatttgggctgcgggtcgtgttccaacgacagataaaattggcttagaaaatgctggcgtagaaacgaacgaacatggctatgtcaaagtagataaatatcaaaatactaatgtgaaaggcatttatgcggtaggcgatattatcgaaaacggcattgaattaacaccagttgcagttgcagcaggtcgtcgcctttctgagcgtttatttaataataaaccgactgaatatttagattacagtttagttccaaccgttgtatttagccatccgcctatcggcactgtaggtttaactgaaccgcaagcgattgagcagtacggcgcagaaaatgttaaggtatataaatcttctttcacagcgatgtacactgcggtaactcaacatcgccaaccgtgcaaaatgaaattagtttgtgtgggtaaagatgaaaaagttgtgggtttacatggtattggttttggtgtagatgaaatgattcaaggatttgctgtagcaatcaaaatgggtgcaacaaaagctgattttgacaatacggtggcaattcatccaacaggttcagaggaatttgtaacaatgcgt

SEQ ID NO: 78 - protein, 456 aa, H. influenzae
MTKHYDYIAIGGGSGGIASLNRAASYGKKCAIIEAKHLGGTCVNVGCVPKKVMFYGAHIAEAINNYAPDYGFDVEVKKFDFSKLIESRQAYISRIHTSYNNVLAKNNIDVINGFGKFVDAHTIEVTLADGTKEQVTADHILIATGGRPYRPNIKGQEYGIDSDGFFALTELPKRAAVIGAGYIAVELSGVLNSLGVETHLLVRRHAPMRNQDPLIVETLVEVLAQDGIQLHTNSTPSEIVKNADGSLTVRCDGQSDVTVDCVIWAAGRVPTTDKIGLENAGVETNEHGYVKVDKYQNTNVKGIYAVGDIIENGIELTPVAVAAGRRLSERLFNNKPTEYLDYSLVPTVVFSHPPIGTVGLTEPQAIEQYGAENVKVYKSSFTAMYTAVTQHRQPCKMKLVCVGKDEKVVGLHGIGFGVDEMIQGFAVAIKMGATKADFDNTVAIHPTGSEEFVTMR

SEQ ID NO: 79 - DNA, 1038 bp, H. influenzae
  CDS (1)...(1038): HI-0172
atgaaaaaattaataagcggtatcatagcggtagcaatggcattaagtcttgctgcttgtcaaaaagaaacaaaagttatctctttaagcggtaaaacaatggggacaacttatcatgttaaataccttgatgatggttcaataacagcaacatctgaaaagacgcatgaagaaattgaagcaatcttaaaagatgtaaacgcgaaaatgtccacttacaaaaaagattcggaattgagccgcttcaatcaaaatacccaagtgaacacaccgattgagatttcagcagattttgccaaagtattagctgaagcgattcgtttaaataaagtgaccgaaggcgcgttggatgtaactgttggccctgtcgtgaatttatggggatttggtcctgaaaaacgccccgaaaagcaacctacaccagaacaattagctgaacgccaagcgtgggttggcattgataaaattaccctagatactaacaaagaaaaagctacattaagtaaagcacttcctcaagtttacgtagatttatcgtcaattgctaaaggctttggcgttgatcaggtggctgaaaagttagaacaattaaatgctcagaattacatggttgaaatcggcggtgaaattcgtgcgaaaggaaaaaatattgaaggtaaaccttggcagattgccattgaaaaaccaactacaacaggcgaaagagcggttgaagcggtcattggattaaacaatatgggaatggcaagttctggcgattaccgtatttactttgaagaaaatggcaaacgctttgcgcacgaaattgatccgaaaacaggttatccaattcagcatcatttagcctcaattacggtacttgcaccaacctcaatgactgcagatggcttatctacagggttatttgtgctgggggaagacaaggcgttagaagtggctgagaaaaataatcttgccgtttatttaatcattagaacagataatggttttgttaccaaatcatcctctgctttcaaaaaattaacagaaacaaaagaa

SEQ ID NO: 80 - protein, 346 aa, H. influenzae
MKKLISGIIAVAMALSLAACQKETKVISLSGKTMGTTYHVKYLDDGSITATSEKTHEEIEAILKDVNAKMSTYKKDSELSRFNQNTQVNTPIEISADFAKVLAEAIRLNKVTEGALDVTVGPVVNLWGFGPEKRPEKQPTPEQLAERQAWVGIDKITLDTNKEKATLSKALPQVYVDLSSIAKGFGVDQVAEKLEQLNAQNYMVEIGGEIRAKGKNIEGKPWQIAIEKPTTTGERAVEAVIGLNNMGMASSGDYRIYFEENGKRFAHEIDPKTGYPIQHHLASITVLAPTSMTADGLSTGLFVLGEDKALEVAEKNNLAVYLIIRTDNGFVTKSSSAFKKLTETKE

SEQ ID NO: 81 - DNA, 1254 bp, H. influenzae
  CDS (1)...(1254): HI-0174
atgaccgcactttatgtgcaaaataaaattaacggcaaaattattttgccgttttctcattcaatgaccaatgtgcttcgccttaaccgaagttacgggagaactatgttaatttcaaatacttataatcaacactttcctcaattgacgcaagagcagcttgcgagaaatgcgacaaaaaaagtcatttgtggtatgtctggtggcgtggattcttctgtgtcagcgtttattcttcaacagcaaggctatcaggtggaaggcctgtttatgaaaaactgggaagaagatgatgatacggattattgtactgccgcagctgatcttgcagatgctcaggctgtatgtgataagttggggatcaaactacataaaattaattttgcggcagaatattgggataatgtctttgagcattttttaaccgaatataaagcagggcgcacgccgaacccagatattttgtgtaataaagaaattaaatttaaagcatttttagaatatgcagctgaagatctcggtgcagattatattgcaacagggcattatgtacgtagagcgggagataatgaaaatgcaaaactattacgtggtttagatcccaataaagatcaaagttattttctttatactttaagccataaacaagtggggcaaagtttattccccgttggtgaaatcgagaagcccattgttcgtgctattgctgaagatcttggcttaattacggcgaagaaaaaagattctacagggatttgttttattggtgagcgtaaatttaaggatttcttagcacgctatttaccagctcaaccaggtaatattcgcactgtagatgatgaaattattggtcgccatgatggattaatgtatcacacattgggacaacgcaaaggattaggcattggtggtctaaaaaatgcgggagatgaagcttggtatgtagtagataaagatgtagaaaataatgaacttattgtcgcacaaggtcacgatcatcctcgtttattttcaaaaggattgattgccagccaattacattgggttgatcgcgaaccaattcgagagtcattacgttgcacggtgaaaacgcgttatcgccaacaagatattccttgtgtgattgaaccaattgatgatgaaaccattcgagttatttttgatgaacctcaatcagcagtaaccccagggcaatctgccgtattttaccttggcgaagtttgtttgggtggcggtattatcgcagaaagaata

SEQ ID NO: 82 - protein, 418 aa, H. influenzae
MTALYVQNKINGKIILPFSHSMTNVLRLNRSYGRTMLISNTYNQHFPQLTQEQLARNATKKVICGMSGGVDSSVSAFILQQQGYQVEGLFMKNWEEDDDTDYCTAAADLADAQAVCDKLGIKLHKINFAAEYWDNVFEHFLTEYKAGRTPNPDILCNKEIKFKAFLEYAAEDLGADYIATGHYVRRAGDNENAKLLRGLDPNKDQSYFLYTLSHKQVGQSLFPVGEIEKPIVRAIAEDLGLITAKKKDSTGICFIGERKFKDFLARYLPAQPGNIRTVDDEIIGRHDGLMYHTLGQRKGLGIGGLKNAGDEAWYVVDKDVENNELIVAQGHDHPRLFSKGLIASQLHWVDREPIRESLRCTVKTRYRQQDIPCVIEPIDDETIRVIFDEPQSAVTPGQSAVFYLGEVCLGGGIIAERI

SEQ ID NO: 83 - DNA, 732 bp, H. influenzae
  CDS (1)...(732): HI-0175
atgcaagcaattaaccccaattggaatgttccaaagaatattcatgcctttaccactactcgtgaagggggtgtgagcttagcgccttatttgagtttcaacttaggcgatcatgtcggtgataacaaaagtgcggtaaaaaccaaccgcactttattagtagaaaaatttggtttgccacaaacacctatatttctaactcaaacacacagtactcgagtgattcaattaccttattcaggacaaaatcttgaagcggatgcagtttatacaaatgttcccaatcaagtttgcgttgttatgacggcagactgtttgccagttctattcactacaacatctggcaatgaagtggctgcaacacatgctggctggcgtggtttatgcgatggtgtactagaagaaacagtgaaatattttcaagctaaacctgaagatattatcgcgtggtttggccctgcaataggtccaaaggcctttcaagttgggattgatgttgtagaaaagtttgttgtagtagatgaaaaagccaaactcgcctttcaacctgatgcaatcgaagaaggtaaatacctgagtaatctttatcaaattgcaactcagcgattaaacaatctaggtattacgcaaatttatggtggaaatcactgtacattcaacgaaaaagaaaagttcttttcttatcgcagggacaatcaaacgggacgaatggcgagcgtcatttggtttgaa

SEQ ID NO: 84 - protein, 244 aa, H. influenzae
MQAINPNWNVPKNIHAFTTTREGGVSLAPYLSFNLGDHVGDNKSAVKTNRTLLVEKFGLPQTPIFLTQTHSTRVIQLPYSGQNLEADAVYTNVPNQVCVVMTADCLPVLFTTTSGNEVAATHAGWRGLCDGVLEETVKYFQAKPEDIIAWFGPAIGPKAFQVGIDVVEKFVVVDEKAKLAFQPDAIEEGKYLSNLYQIATQRLNNLGITQIYGGNHCTFNEKEKFFSYRRDNQTGRMASVIWFE

SEQ ID NO: 85 - DNA, 738 bp, H. influenzae
  CDS (1)...(738): HI-0179
atgtcagttcttggacgaattcactcttttgaatcctgtggcactgtagatgggccaggtattcgttttattttatttatgcaaggctgcttgatgcgctgcaaatattgccacaatcgtgatacttgggatcttgaaggtggtaaagaaatcagtgtcgaagatttaatgaaagaagtcgtgacttatcgccattttatgaatgctactggcggtggtgtcacagcatctggtggcgaggctgtgttacaagcagagtttgtacgcgattggttccgtgcttgtaaagaggaagggattaatacttgcttagatacaaatggttttgtacgtcattatgatcatattattgatgaattattagatgtaacagatcttgttttacttgatttaaaagaacttaatgatcaagttcatcaaaatcttattggggtgccaaataaacgtacccttgaatttgcaaaatatttgcaaaaacgtaatcaacatacctggattcgttatgttgtggttcctggttatactgatagcgatcacgatgtgcatttattaggtcagtttattgaaggtatgaccaatattgaaaaagttgaacttcttccttatcatcgattaggtgtgcataaatggaaaacccttgggttagattatgagcttgaaaatgtattaccgccaactaaagaatccttagaacatattaaaacaatcctagaaggttatggacacactgtaaaattc

SEQ ID NO: 86 - protein, 246 aa, H. influenzae
MSVLGRIHSFESCGTVDGPGIRFILFMQGCLMRCKYCHNRDTWDLEGGKEISVEDLMKEVVTYRHFMNATGGGVTASGGEAVLQAEFVRDWFRACKEEGINTCLDTNGFVRHYDHIIDELLDVTDLVLLDLKELNDQVHQNLIGVPNKRTLEFAKYLQKRNQHTWIRYVVVPGYTDSDHDVHLLGQFIEGMTNIEKVELLPYHRLGVHKWKTLGLDYELENVLPPTKESLEHIKTILEGYGHTVKF

SEQ ID NO: 87 - DNA, 825 bp, H. influenzae
  CDS (1)...(825): HI-0184
atgaaactaattgaacaacatcaaatttttggcggttcgcagcaagtttgggcgcataatgctcaaacacttcaatgtgaaatgaaatttgccgtttatttgccaaataatccagaaaatcgaccgcttggtgtgatttattggctttcaggcttaacttgtactgagcaaaatttcattaccaaatcaggcttccagcgttacgcggcagaacatcaagtgattgttgttgctcccgatacaagccctcgtggagagcaagtgccgaatgatgcggcttacgatttagggcagggagcgggcttttatcttaatgcgaccgagcagccttgggcgacgaattatcaaatgtatgattatatcttgaatgaattgcctgatttgattgaagcaaatttccctaccaacggcaaacgttccattatgggacattcaatgggtggacacggtgcattggtattggcactgcgaaatcgggaacgttatcaaagcgtttctgccttttcgcccattttgtcgccaagccttgtgccttggggagaaaaagccttttctgcctatttaggggaagatcgtgaaaaatggcagcaatatgatgccagctcgctcattcaacaaggctataaagtgcaaggtatgcgcattgatcagggcttggaagatgagtttttaccgacacaattgcgtaccgaagattttatagaaacctgtcgagtggcaaatcagccagtcgatgtgcgcttccataaaggctatgatcacagctattacttcatcgccagttttattggcgagcatattgcctatcatgcggaatttttgaag

SEQ ID NO: 88 - protein, 275 aa, H. influenzae
MKLIEQHQIFGGSQQVWAHNAQTLQCEMKFAVYLPNNPENRPLGVIYWLSGLTCTEQNFITKSGFQRYAAEHQVIVVAPDTSPRGEQVPNDAAYDLGQGAGFYLNATEQPWATNYQMYDYILNELPDLIEANFPTNGKRSIMGHSMGGHGALVLALRNRERYQSVSAFSPILSPSLVPWGEKAFSAYLGEDREKWQQYDASSLIQQGYKVQGMRIDQGLEDEFLPTQLRTEDFIETCRVANQPVDVRFHKGYDHSYYFIASFIGEHIAYHAEFLK

SEQ ID NO: 89 - DNA, 1347 bp, H. influenzae
  CDS (1)...(1347): HI-0189
atgtcaaaagttgcttccttagacgcatttttaacaaaagttgctcaacgcgatggttatcaacctgaatttttacaagcggttcgcgaggtattcacatcaatttggccttttttagaagccaatcctaaatatcgttcagaagcattattagaacgtttagttgagcctgaacgtgcatttcaattccgtgtggcgtggactgacgataaagggcaagtgcaagtaaacagagcatttcgtgtacaatttaatagtgccataggcccatttaaagggggaatgcgtttccatccatcagtaaatttatctatcttaaaattcttaggttttgagcaaatctttaaaaatgctttaacaacattgcctatgggcggggcaaaaggcggttcagattttgatcctaaaggcaaatctgatgctgaagttatgcgtttttgccaagcattaatggctgaactttatcgtcacgtaggagctgatacagatgttcccgcaggcgatataggcgtcggtgggcgcgaagttggctatttagctggctatatgaaaaaattatcaaaccaatcagcctgtgttttcactggtcgcggtctttctttcggtggtagtttaattcgtccggaagcaacgggatatggattaatttattttgctcaagcaatgcttgctgaaaaaggcgatagttttgcaggtaaagtagtttcagtttctggttctggtaatgtagcacaatatgctattgaaaaagcattatctcttggtgcaaaagtagtaacttgttctgattcatcaggttatgtttatgatccaaatggatttactactgaaaaattagccgcacttttcgatattaaaaatacaaaacgtgggcgtgtgaaagattatgcagaacagtttggtttgcaatattttgaaggtaaacgcccttgggaagtgcaagttgatattgcgcttccttgtgcaactcaaaatgaattagaactttctgatgcacaacgtttaattaaaaatggtgtgaaattagtggctgaaggtgcgaatatgccaacaacaattgaagcaacagaagcattactagctgcagatgtattatttggcccgggtaaagctgccaacgctggtggtgttgctacttctggtttagaaatggcacaaagttcacaacgtttatattggacagcggaagaagtggacgctcaattacatcgcattatgttagatattcacgcaaactgtaaaaaatacggcacaattgaaggtcaagaaaacattaactatgttgttggggcaaatgtagcaggctttgttaaggtggctgatgcaatgttagcccaaggcgtttat

SEQ ID NO: 90 - protein, 449 aa, H. influenzae
MSKVASLDAFLTKVAQRDGYQPEFLQAVREVFTSIWPFLEANPKYRSEALLERLVEPERAFQFRVAWTDDKGQVQVNRAFRVQFNSAIGPFKGGMRFHPSVNLSILKFLGFEQIFKNALTTLPMGGAKGGSDFDPKGKSDAEVMRFCQALMAELYRHVGADTDVPAGDIGVGGREVGYLAGYMKKLSNQSACVFTGRGLSFGGSLIRPEATGYGLIYFAQAMLAEKGDSFAGKVVSVSGSGNVAQYAIEKALSLGAKVVTCSDSSGYVYDPNGFTTEKLAALFDIKNTKRGRVKDYAEQFGLQYFEGKRPWEVQVDIALPCATQNELELSDAQRLIKNGVKLVAEGANMPTTIEATEALLAADVLFGPGKAANAGGVATSGLEMAQSSQRLYWTAEEVDAQLHRIMLDIHANCKKYGTIEGQENINYVVGANVAGFVKVADAMLAQGVY

SEQ ID NO: 91 - DNA, 438 bp, H. influenzae
  CDS (1)...(438): HI-0190
atgtctgaaggaaatattaaattactcaaaaaagtgggattaaaaattacagagcctcgcttaactattctcgctttaatgcaaaatcataaaaatgaacatttttctgcagaagatgtttataaaattttcctggaacaaggttgtgaaattggattagccacagtttatcgtgtgcttaatcaatttgatgaagcgcatattgtaatccgtcataattttgagggaaataaatccgtttttgagcttgctccaacagaacatcacgatcatattatttgtgaagattgcggtaaagtatttgaatttacggataatattattgaacaacgtcagcgtgaaatcagtgaaaaatacggcataaaattaaaaacgcataacgtgtatctttacggcaaatgcagtgatattaatcattgtgacgaaaacaattcaaaa

SEQ ID NO: 92 - protein, 146 aa, H. influenzae
MSEGNIKLLKKVGLKITEPRLTILALMQNHKNEHFSAEDVYKIFLEQGCEIGLATVYRVLNQFDEAHIVIRHNFEGNKSVFELAPTEHHDHIICEDCGKVFEFTDNIIEQRQREISEKYGIKLKTHNVYLYGKCSDINHCDENNSK

SEQ ID NO: 93 - DNA, 522 bp, H. influenzae
  CDS (1)...(522): HI-0191
atggcaatagttggtcttttttatggtagtgatactggaaatactgaaaacatcgcaaaacaaatccaaaaacaattaggtagtgatttaattgatattcgtgatattgccaaaagtagcaaagaagatattgaagcatacgatttcttgcttttcggtatcccaacttggtattacggcgaagcacaagcagactgggatgactttttcccaacactcgaagaaattgattttacagataaacttgtaggtattttcggttgtggcgatcaagaagattatgcagattatttctgtgatgctatcggaactgtgcgcgatattatagagccacacggtgcaattgtggtaggaaattggccaacagaaggctataattttgaagcttcgaaagccttattggaagatggcactttcatcggattatgtattgatgaagatcgccaaccagagcttaccgcagagcgtgtagaaaaatggtgtaaacaaatttatgatgaaatgtgcttagctgaattggct

SEQ ID NO: 94 - protein, 174 aa, H. influenzae
MAIVGLFYGSDTGNTENIAKQIQKQLGSDLIDIRDIAKSSKEDIEAYDFLLFGIPTWYYGEAQADWDDFFPTLEEIDFTDKLVGIFGCGDQEDYADYFCDAIGTVRDIIEPHGAIVVGNWPTEGYNFEASKALLEDGTFIGLCIDEDRQPELTAERVEKWCKQIYDEMCLAELA

SEQ ID NO: 95 - DNA, 1356 bp, H. influenzae
  CDS (1)...(1356): HI-0194
atgtatccttggcaagattttgctattcaacctgatttctcagataaaatcgctttgcgtactacgcagggcgatatgcttacttggatagaattgactacaaagattaaccaaacagtggcttttttacaaaaaaaaggcgtaaatgcagaaagtgcggttgcttttgtgggaaaaaattcagagaaaattttatttttatatctggcgacaattcagcttggcgcaaaagttttaggcataaaccctgcttttccacaagaaaaaattgcaaaattatgtgagttttatcaaattgatttttgtttttatgataaagatttactgaatttgcaagaaattgatgtttttactcaaaaagccgatttttttcgtcctgcgacgatgacgctaacgtctggctcgacaggtttaccaaaagcagttgtgcataatgtccaagcacatttggataatgcaaaaggggtatgtaacttaatgaagtttgattgtaatcaatcttggttactttcattacccttatatcacgtttcagggcaaggtattgtttggcgttggttatattgcggtgcacaattacatttcccagaagatgatttttatgcttcattattaaagacgacccacgtttctcttgtgccaacgcaattacagcgtttattagattatttacaggaaaatccgagcatttcatttgctacacgccatattttactgggcggtgcgcatattccgacagaacttacacaaaatatgttgaaatatggtatcgaaacgtattctggctacggaatgacggaaatggcttcgacagtttttgctaaaaaatctgacagaaaacaaggcgtagggcaaccgctcttaggtagagagtattgtttagtaaatgatgaaatttggctgaaaggtgcaggtttggcgatgggttattggaaagatcgacaaattgttccattaacgaataaccaaggctggattcagacaaaagataaaggcatttggcaagagggcgaacttgttattatcggacgacttgataatatgtttatttcaggtggcgaaaatattcagccagaagaaattgaacaagtgattattcaacattcttcagttaatcaagtatttgttttaccacaaaaaaacaaagaatttggtcagcgtcctgtcgctttagtggattttaatgagccctttagcaaaagtgcggttgaaaatttaatgttttttttacaagataaacttgcacgttttaaacaacctattgcatattatcctttgccactgatgcttgagaaaggcattaagatctcacgtaaacagcttgctgattggttggcaaagcgagatgagataaat

SEQ ID NO: 96 - protein, 452 aa, H. influenzae
MYPWQDFAIQPDFSDKIALRTTQGDMLTWIELTTKINQTVAFLQKKGVNAESAVAFVGKNSEKILFLYLATIQLGAKVLGINPAFPQEKIAKLCEFYQIDFCFYDKDLLNLQEIDVFTQKADFFRPATMTLTSGSTGLPKAVVHNVQAHLDNAKGVCNLMKFDCNQSWLLSLPLYHVSGQGIVWRWLYCGAQLHFPEDDFYASLLKTTHVSLVPTQLQRLLDYLQENPSISFATRHILLGGAHIPTELTQNMLKYGIETYSGYGMTEMASTVFAKKSDRKQGVGQPLLGREYCLVNDEIWLKGAGLAMGYWKDRQIVPLTNNQGWIQTKDKGIWQEGELVIIGRLDNMFISGGENIQPEEIEQVIIQHSSVNQVFVLPQKNKEFGQRPVALVDFNEPFSKSAVENLMFFLQDKLARFKQPIAYYPLPLMLEKGIKISRKQLADWLAKRDEIN

SEQ ID NO: 97 - DNA, 954 bp, H. influenzae
  CDS (1)...(954): HI-0199
atgtcggataatcaacaaaatttacgtttgacggcgagagtgggctatgaagcgcacttttcatggtcgtatttaaagcctcaatattgggggatttggcttggtattttctttttattgttgttagcatttgtgccttttcgtctgcgcgataaattgacgggaaaattaggtatttggattgggcataaagcaaagaaacagcgtacgcgtgcacaaactaacttgcaatattgtttccctcattggactgaacaacaacgtgagcaagtgattgataaaatgtttgcggttgtcgctcaggttatgtttggtattggtgagattgccatccgttcaaagaaacatttgcaaaaacgcagcgaatttatcggtcttgaacatatcgaacaggcaaaagctgaaggaaagaatattattcttatggtgccacatggctgggcgattgatgcgtctggcattattttgcacactcaaggcatgccaatgacttctatgtataatccacaccgtaatccattggtggattggctttggacgattacacgccaacgtttcggcggaaaaatgcatgcacgccaaaatggtattaaaccttttttaagtcatgttcgtaaaggcgaaatgggttattacttacccgatgaagattttggggcggaacaaagcgtatttgttgatttctttgggacttataaagcgacattaccagggttaaataaaatggcaaaactttctaaagccgttgttattccaatgtttcctcgttataacgctgaaacgggcaaatatgaaatggaaattcatcctgcaatgaatttaagtgatgatcctgaacaatcagcccgagcaatgaacgaagaaatagaatcttttgttacgccagcgccagagcaatatgtttggattttgcaattattgcgtacaaggaaagatggcgaagatctttatgat

SEQ ID NO: 98 - protein, 318 aa, H. influenzae
MSDNQQNLRLTARVGYEAHFSWSYLKPQYWGIWLGIFFLLLLAFVPFRLRDKLTGKLGIWIGHKAKKQRTRAQTNLQYCFPHWTEQQREQVIDKMFAVVAQVMFGIGEIAIRSKKHLQKRSEFIGLEHIEQAKAEGKNIILMVPHGWAIDASGIILHTQGMPMTSMYNPHRNPLVDWLWTITRQRFGGKMHARQNGIKPFLSHVRKGEMGYYLPDEDFGAEQSVFVDFFGTYKATLPGLNKMAKLSKAVVIPMFPRYNAETGKYEMEIHPAMNLSDDPEQSARAMNEEIESFVTPAPEQYVWILQLLRTRKDGEDLYD

SEQ ID NO: 99 - DNA, 348 bp, H. influenzae
  CDS (1)...(348): HI-0201
atgtctaacattatcaaacaacttgaacaagaacaattaaaacaaaacgtacctagcttccgcccaggtgatactttagaagttaaagtatgggtggttgaaggtagcaaacgtcgtttgcaagcattcgaaggcgtggttattgcaattcgtaaccgtggcttgcactcagcatttactttacgtaaagtatctaacggcgtaggcgttgagcgtgtattccaaactcactctccagctgtagattctatcgcagttaaacgtaaaggtgcggtacgtaaagctaaactttactacttacgtgaacgttcaggtaaatcagctcgtattaaagagcgtttaggcgca

SEQ ID NO: 100 - protein, 116 aa, H. influenzae
MSNIIKQLEQEQLKQNVPSFRPGDTLEVKVWVVEGSKRRLQAFEGVVIAIRNRGLHSAFTLRKVSNGVGVERVFQTHSPAVDSIAVKRKGAVRKAKLYYLRERSGKSARIKERLGA

SEQ ID NO: 101 - DNA, 534 bp, H. influenzae
  CDS (1)...(534): HI-0203
gtgaaaaatatggaacaacaacatattgaagttgtgggcaaattaggctcaacctacggtattcgtgggtggttgcgtatttattcatcaacagaacaagctgaaagcatttttgattatcaaccttggtttttaaaaatcaaaggcgaatggcaatcaattgaattagaaaactggcgttatcataatcacgaaatcatcgttaaattaaaaggcgttgatgaccgtgaagctgcacaaattttagcgaatgttgaaattggtgtagatttatctgttttcccagaactagaagagggcgattattactggcacgatttaatcggttgtacagtcgtaaacttagaaggttatacaatgggaacagtaacagaaatgatggaaacgggttctaatgatgtattagtggttaaagccaataccaaagatgcttttggaaaacaagagcggttaattccgtttttgtatgaacaagtagttaaaagagtcgatctcaccacgaaaactattgaagtggattgggacgctggtttc

SEQ ID NO: 102 - protein, 178 aa, H. influenzae
VKNMEQQHIEVVGKLGSTYGIRGWLRIYSSTEQAESIFDYQPWFLKIKGEWQSIELENWRYHNHEIIVKLKGVDDREAAQILANVEIGVDLSVFPELEEGDYYWHDLIGCTVVNLEGYTMGTVTEMMETGSNDVLVVKANTKDAFGKQERLIPFLYEQVVKRVDLTTKTIEVDWDAGF

SEQ ID NO: 103 - DNA, 1809 bp, H. influenzae
  CDS (1)...(1809): HI-0206
atgcttttatccaaaaaatcagcctcctttgcactcagtgcatttgcgatgcttttcactagtgtagctcttgccaaagaggcaccacaagctcacaaagctgtggaattaagtattttgcatatcaatgatcaccattcttatttagaaccgcacgaaacacggattaatttaaatggtcagcaaaccaaagtggatattggtggtttttctgctgtcaatgcaaaacttaacaaattgcgtaaaaaatacaaaaatccattagtactgcatgcaggcgatgccattactggtacactttacttcacgctgtttggtggttctgcagatgcagctgtgatgaatgcaggtaatttccattattttactttaggtaatcatgaatttgacgcgggtaatgaagggttattaaaactgcttgaaccattaaaaatcccagtgctttcagctaatgtgattcctgataaaaattcaattttgtataacaaatggaaaccttacgatattttcactgtggatggagaaaaaattgccattatcggtttagatactgtgaataaaacagtgaattcctcttctccgggtaaggatgtgaagttctacgatgaaattgctactgcacaaattatggcaaatgcgctaaaacagcaaggaattaataaaattatcctactttcacacgcaggtagtgaaaaaaatatcgaaattgctcaaaaagtaaatgatattgatgtgatcgttactggcgattcacattatttatacggaaatgatgaattacgtagtttaaaacttccagtaatctatgaatatccacttgaatttaaaaatccaaatggagaccctgtatttgtaatggaaggctgggcttattctgccgtggtgggggatttgggtgttaaattcagtcctgaaggtatagcgtctattactcgtaaaattcctcatgtgttaatgagttctcataaacttcaagtgaaaaatgcggaaggtaaatggacggaattaactggcgatgaacgtaaaaaagcacttgatactttaaaatcaatgaagagtatttcacttgatgatcatgatgcaaaaacagacatgcttatttcaaaatataaaagtgaaaaagatcgtttagcacaagaaattgttggtgtcatcacaggttctgcaatgccgggtggttcagcaaaccgtatcccaaataaagcaggctcaaatccagaaggttctattgcaacgcgttttattgcagaaacaatgtataacgaactcaaaacagtggatttaactattcaaaatgctggcggtgtacgcgcagatattttaccgggtaatgtaacctttaacgatgcttatactttcttacctttcgggaatacgttatatacctataaaatggaaggttcattagtgaaacaagtgcttgaagatgcaatgcaatttgctttggttgatggctctacaggcgcattcccttatggtgcaggtattcgttatgaagcgaatgaaacaccaaatgcggaaggtaagcgtttagtgagtgttgaagtcttgaataaacaaacccaacaatgggaaccgattgatgataacaaacgttatcttgtcggaacaaatgcttatgttgcaggcggtaaagacggttataaaacctttggtaaattatttaacgatccaaaatatgaaggcgttgatacctacttacctgatgcagaaagtttcataaaatttatgaaaaaacatccgcactttgaggcttacacttcatcaaatgtgaaatttaatgcttcaactgatgcattacctaaaaaa

SEQ ID NO: 104 - protein, 603 aa, H. influenzae
MLLSKKSASFALSAFAMLFTSVALAKEAPQAHKAVELSILHINDHHSYLEPHETRINLNGQQTKVDIGGFSAVNAKLNKLRKKYKNPLVLHAGDAITGTLYFTLFGGSADAAVMNAGNFHYFTLGNHEFDAGNEGLLKLLEPLKIPVLSANVIPDKNSILYNKWKPYDIFTVDGEKIAIIGLDTVNKTVNSSSPGKDVKFYDEIATAQIMANALKQQGINKIILLSHAGSEKNIEIAQKVNDIDVIVTGDSHYLYGNDELRSLKLPVIYEYPLEFKNPNGDPVFVMEGWAYSAVVGDLGVKFSPEGIASITRKIPHVLMSSHKLQVKNAEGKWTELTGDERKKALDTLKSMKSISLDDHDAKTDMLISKYKSEKDRLAQEIVGVITGSAMPGGSANRIPNKAGSNPEGSIATRFIAETMYNELKTVDLTIQNAGGVRADILPGNVTFNDAYTFLPFGNTLYTYKMEGSLVKQVLEDAMQFALVDGSTGAFPYGAGIRYEANETPNAEGKRLVSVEVLNKQTQQWEPIDDNKRYLVGTNAYVAGGKDGYKTFGKLFNDPKYEGVDTYLPDAESFIKFMKKHPHFEAYTSSNVKFNASTDALPKK

SEQ ID NO: 105 - DNA, 1155 bp, H. influenzae
  CDS (1)...(1155)
atgaaaaacaaccgcacttttttagaaaaattattggaggggtctgaggttgagtggaagcctttagatgaagttgccaatatcgtcaataatgcaagaaaaccagtcaaatcatccttacgagtgtcagggaatattccatattatggtgctaataatatccaagattatgtcgaaggatatactcacgagggcgaatttgtattaattgctgaagatggttctgctagtctagaaaattactctattcaatgggctgtaggtaaattttgggcaaataaccacgtccatgtagtaaatggcaaagaaaaattaaataatcgttttttataccattatttaactaatatgaatttcattccattcttagctggaaaggaacgtgcaaaattaacaaaagctaaactgcaacaaattccaatccccatccccccactctccgtccaaaccgaaatcgtaaaaattttggacgcattgacagcacttaccagcgagcttaccagcgagcttaccagcgagcttaccagcgagcttatactacgccagaaacagtatgaatactatcgagaaaaattgttaaatattgatgaaatgaacaaggttattgaacttggtgatgtagggcctgttcgtatgtgtaaacgaattcttaaaaaccaaacagcaagctctggagatattccattttataagattggaacttttgggaaaaagcctgatgcctatatttcaaatgagcttttccaagaatataaacaaaaatatagctatcctaaaaaaggagatattcttatatctgctagcggaactattggtagaacagttatatttgatggagaaaattcttattttcaagatagcaatatcgtctggattgataatgatgaaactctcgttcttaataaatatttatatcatttctataagattgctaaatggggaattgcagaaggaggcaccattcaacgtttatataacgacaatttgaaaaaagtaaaaatttctattcctccattaaaggaacaacaccgcatcgtttcaatcctagataaatttgaaaccttaaccaattccatcactgaaggcttacctttagcgattgaacaaagccaaaagcggtatgaatattaccgagaattgctattaaatttctcg

SEQ ID NO: 106 - protein, 385 aa, H. influenzae
MKNNRTFLEKLLEGSEVEWKPLDEVANIVNNARKPVKSSLRVSGNIPYYGANNIQDYVEGYTHEGEFVLIAEDGSASLENYSIQWAVGKFWANNHVHVVNGKEKLNNRFLYHYLTNMNFIPFLAGKERAKLTKAKLQQIPIPIPPLSVQTEIVKILDALTALTSELTSELTSELTSELILRQKQYEYYREKLLNIDEMNKVIELGDVGPVRMCKRILKNQTASSGDIPFYKIGTFGKKPDAYISNELFQEYKQKYSYPKKGDILISASGTIGRTVIFDGENSYFQDSNIVWIDNDETLVLNKYLYHFYKIAKWGIAEGGTIQRLYNDNLKKVKISIPPLKEQHRIVSILDKFETLTNSITEGLPLAIEQSQKRYEYYRELLLNFS

SEQ ID NO: 107 - DNA, 528 bp, H. influenzae
  CDS (1)...(528): HI-0217
atgtctaattatcggcgcgattttactaaaggtggattatattttttcacaattgttttacaagatagaacaaaatcttatctaactgactatatcaatgaatttagatctgcatataaacaaacttgtgaacattatccattcgaaacagtagcaatttgtattttgcccgatcatattcatttactgatgcaattacctgaaaatgatgataattacgcaatacgcatcgcatatttaaaaacacaatttacacgacaacttccaaaagaatgccgacaatttaataaaaatagacaaaaatatcgagaatcaggtatttggcaacgccgattttgggagcatttaattcgtgatgataaagatttagcgaatcatttagattatatttattacaatcctgtgaaacacggctatgttgaggtagtaaaagattggccgtattcttccttccatcgtgatgtgaaagcagagatttatcctgaagattggggaggcaacccagatttgaaaattaaaggtgatata

SEQ ID NO: 108 - protein, 176 aa, H. influenzae
MSNYRRDFTKGGLYFFTIVLQDRTKSYLTDYINEFRSAYKQTCEHYPFETVAICILPDHIHLLMQLPENDDNYAIRIAYLKTQFTRQLPKECRQFNKNRQKYRESGIWQRRFWEHLIRDDKDLANHLDYIYYNPVKHGYVEVVKDWPYSSFHRDVKAEIYPEDWGGNPDLKIKGDI

SEQ ID NO: 109 - DNA, 906 bp, H. influenzae
  CDS (1)...(906): HI-0220.1
atgatgaattttacgttattgacatatttagcggattgtcagccgaaagtgcggtctgaattggaaaaattttctaaaaatctagaggaagatattcaacaattacgtgaaattggtttggatattctcgttgatggacaagattatcgcttagtgccaatgcttcctttattaaatcctcagcaaatttcaaccgcactttttccttatagtatccattatcagccgattatttcctctacaaatgaatggatactacaaaatattctttcattaaaaaaaggcgatctttgtgtggctgaatatcaaactgcaggacgaggtcggcgtggtcgccaatggttatcgccttttgccgggcaaatcatgtttagtttttattgggcatttgatcctaaaaaatcaattgaggggttaagtttagtcatcggtttggcgattgcggaagtgctaaatgtacaagtgaaatggccaaatgatattttgtttgatgaaagaaagttaggtggcattttagttgaaattgctaatcataaaaatggcatgctcaatttagtgattggcataggtattaatgtgtcgttgtcaaaacaaacagaaattagtcagccttatgcggaagtgtgtgaaattgatcctgatgtagagcgacaaactttattacccaaacttatacaacatttatatacacgtttaaatatctttgaacaaaatggtattgatgaggaatttcaacaagcatggcaatcatataatgcgttttcaaatagtgaaataaatgtgcttactgagcaaggcgttatttcaggtattgaacaaggcatagatgaacgtggttatttaaaagtattatgtggcaataaaattcagatgtttaatggtggagaagtttcattacgtaagaaa

SEQ ID NO: 110 - protein, 302 aa, H. influenzae
MMNFTLLTYLADCQPKVRSELEKFSKNLEEDIQQLREIGLDILVDGQDYRLVPMLPLLNPQQISTALFPYSIHYQPIISSTNEWILQNILSLKKGDLCVAEYQTAGRGRRGRQWLSPFAGQIMFSFYWAFDPKKSIEGLSLVIGLAIAEVLNVQVKWPNDILFDERKLGGILVEIANHKNGMLNLVIGIGINVSLSKQTEISQPYAEVCEIDPDVERQTLLPKLIQHLYTRLNIFEQNGIDEEFQQAWQSYNAFSNSEINVLTEQGVISGIEQGIDERGYLKVLCGNKIQMFNGGEVSLRKK

SEQ ID NO: 111 - DNA, 900 bp, H. influenzae
  CDS (1)...(900): HI-0223
atgaagttcaagatgaaagtagtcactcaaggcatattattgtgcattttttcacaatgtttatttggcatattgtatttatttagtatatggctccaaccacttagcggaacggatgtttttgcgtggcgtatgctgaccatgatctttgggttacttttaattctatttccgactattggttgccgctctcttttgtctcttattacgacaactttaggaaaaagttggacgcgttgggttttatttttactggggacactagatgcgggaagtcaattctggctctttatgtgggcaccgctaaatggcgaagggataaatattgcgatgggatattttctttttccgctgattatggctgttttaggatgggcttggttaaaagaacgtttatcatttattcagaaaatcgcgcttttactggctgctgccggtgttgcacatgaattatggcacactcaaagtttttcatggacaagcttgtgggtttgtacggtttatccattttattatctaagccgtaaatggatgaaaatccctgctctacaaggaattacattggatattattttaatttcgattccttgttttatttatattctttctcaaagcgatacgctttctttggttacgcaagaatatcgttattggttattgctaccagcacttggtattgtgagtgcgatttcgctttcagccaacttaaaatcaagccaacaaattccagttagtatttttgcggtgttgagttatattgaacccatattgctttttttaatagctgtctttgtgctagataatcaaataaccacaagtgactattttacttatgtgcctatttggttaagtctcattgtgattggcatagaagggcttttaaacaaaaagaaagtgcgg

SEQ ID NO: 112 - protein, 300 aa, H. influenzae
MKFKMKVVTQGILLCIFSQCLFGILYLFSIWLQPLSGTDVFAWRMLTMIFGLLLILFPTIGCRSLLSLITTTLGKSWTRWVLFLLGTLDAGSQFWLFMWAPLNGEGINIAMGYFLFPLIMAVLGWAWLKERLSFIQKIALLLAAAGVAHELWHTQSFSWTSLWVCTVYPFYYLSRKWMKIPALQGITLDIILISIPCFIYILSQSDTLSLVTQEYRYWLLLPALGIVSAISLSANLKSSQQIPVSIFAVLSYIEPILLFLIAVFVLDNQITTSDYFTYVPIWLSLIVIGIEGLLNKKKVR

SEQ ID NO: 113 - DNA, 372 bp, H. influenzae
  CDS (1)...(372): HI-0228
gtgatgctaattatcttccttaatgtagaaattacactgaaatctttattaatgcacaatgaaaatctctctgtttttattttacatacaggagatatttctgaatcgtggcaaaatgatcttcaactttattttgctaaacgctattcaacattacaattagttcatatgatttcaattaatactcttgatacttctcctaatatttttcattttacaggtcctcataagcctcttgataatattttttctgaaaatgcttgtgttaatgccgtaatttcattgtttagactttatgcgtcaataagttggcaagatatttgctctcttccactcggtactacaagagctaattggattaatcaagagaga

SEQ ID NO: 114 - protein, 124 aa, H. influenzae
VMLIIFLNVEITLKSLLMHNENLSVFILHTGDISESWQNDLQLYFAKRYSTLQLVHMISINTLDTSPNIFHFTGPHKPLDNIFSENACVNAVISLFRLYASISWQDICSLPLGTTRANWINQER

SEQ ID NO: 115 - DNA, 144 bp, H. influenzae
  CDS (1)...(144): HI-0234
gtgcgtaaagtggaggatcaaataaaaattcagcgttcttttacagagttaaatgaattgttcaaatttttaggggattattttgatcccgtctcgattggtctagtcggtgtgaaaattggaaacttagggataaaattagaa

SEQ ID NO: 116 - protein, 48 aa, H. influenzae
VRKVEDQIKIQRSFTELNELFKFLGDYFDPVSIGLVGVKIGNLGIKLE

SEQ ID NO: 117 - DNA, 348 bp, H. influenzae
  CDS (1)...(348): HI-0236
atgtctgttattatttatcacaacccacattgctcaaaaagccgtgaaacgctagcattattagaaaataaaggtattcagccgattattgaactgtatttgcaaaagcagtattccgttaatgaattacaaagcattgccaaaaaattgggaattgatgatgttcgccaaatgatgcgcacgaaagatgaactatataaaagcttaaatttagataatttagatctttctcaagcagaattatttaaagcgataagtgaacattcagcacttattgaacgcccaattgttattaatggcgataaagctaaaatcgggcgtccaccagaaactgtacttgagattttg

SEQ ID NO: 118 - protein, 116 aa, H. influenzae
MSVIIYHNPHCSKSRETLALLENKGIQPIIELYLQKQYSVNELQSIAKKLGIDDVRQMMRTKDELYKSLNLDNLDLSQAELFKAISEHSALIERPIVINGDKAKIGRPPETVLEIL

SEQ ID NO: 119 - DNA, 1848 bp, H. influenzae
  CDS (1)...(1848): HI-0240
atgttaaaccgttatccattatggaagaatttgatggttatttttattgtggccatcgggattttatattctcttccaaatatttatggtgaagatcctgcggtgcaaatttccggtacacgcggtcaagaagcaaatactagcgtgcttggacaagttcaagatgtgcttaaaaccaataatcttccaaccaaatctatcgtgcttgagaatggctcaattctagctcgttttactaataccgatgatcaacttcttgctaaagataaaattgctgaacgtcttggcaataattacaccaccgcattaaatcttgctccagccactccagcttggttaagtatgtttggtgcgaatcctatgaaatggggattagacttacgcggtggggttcgttttttgatggaagtcgatatgaatgccacacttgtaaaacgccaagagcaattacaagacagtttgcgtggcgaacttcgtaaagaaaaaattcaatatactgccattaaaaatactgagcattttggtacgttgataaccttagctaatgtgagccagcgtgctaaagctgagcgaattattcgccaattacatccaacattagatattactgagcctgatgctgatagtattaatttagggctatctactgcagcattaaatgaagcacgcgacttagccattgagcaaaacttaacgattttacgtaaacgtgttgctgaattaggtgttgcagaagcggtaattcaacgtcaaggtgcggagcgtattgtgattgaattaccaggtgttcaagacactgcacgtgcaaaagaaattttaggggcaacggcaacacttgagtttcgtatcgtaaatcaaaatgttacggctgatgctatttctcgtaatatgttaccagctgattcggaagttaaatatgatcgccaaggtcatcctgttgcattatttaaacgtgccgtattaggcggggagcatattattaattcaagctctggtttagatcagcattcaagcacgccacaagtgagtgtaaccttggatagcgaaggtggcgagattatgtctcagaccactaaaaaatattacaagaaaccaatggcaacgctttatgttgaatataaagataacggtaaaaaagatgaaaatggtaaaactattttagaaaagcatgaagaagtgattaatgttgcaacaattcaaggacgttttggttctaatttccaaattactggtgttgatagcattgcggaagcacataatctttctaccttattgaaatctggtgcattaattgcaccaattcaaattgttgaagaacgcacaattggcccatcattaggtgcgcaaaacgtagagcaagggattaatgcgagtctttggggattagttgctgttattgcctttatgttgttttactacaaaatgtttggtgtgattgcaagttttgcacttgttattaatatcgtattacttgtgggattaatgtctattttacccggcgcgacactttcaatgccgggtattgcgggtatcgttttaactttaggtatgtcagtagatgcgaatgtattgatttttgaacgtattaaagaagaaattcgtaatggtcgttcaattcagcaagccattaatgaaggttataacggcgcatttacttctatttttgatgcaaacttaaccacaatcttaaccgcaattattctatacgcggtaggaacaggcccaattcaaggttttgcgattacgctttcacttggtgttgcgatttctatgtttaccgcgattacaggaactcgcgcattagttaatgccctttacggtggtaaacaacttaaaaaattattaatt

SEQ ID NO: 120 - protein, 616 aa, H. influenzae
MLNRYPLWKNLMVIFIVAIGILYSLPNIYGEDPAVQISGTRGQEANTSVLGQVQDVLKTNNLPTKSIVLENGSILARFTNTDDQLLAKDKIAERLGNNYTTALNLAPATPAWLSMFGANPMKWGLDLRGGVRFLMEVDMNATLVKRQEQLQDSLRGELRKEKIQYTAIKNTEHFGTLITLANVSQRAKAERIIRQLHPTLDITEPDADSINLGLSTAALNEARDLAIEQNLTILRKRVAELGVAEAVIQRQGAERIVIELPGVQDTARAKEILGATATLEFRIVNQNVTADAISRNMLPADSEVKYDRQGHPVALFKRAVLGGEHIINSSSGLDQHSSTPQVSVTLDSEGGEIMSQTTKKYYKKPMATLYVEYKDNGKKDENGKTILEKHEEVINVATIQGRFGSNFQITGVDSIAEAHNLSTLLKSGALIAPIQIVEERTIGPSLGAQNVEQGINASLWGLVAVIAFMLFYYKMFGVIASFALVINIVLLVGLMSILPGATLSMPGIAGIVLTLGMSVDANVLIFERIKEEIRNGRSIQQAINEGYNGAFTSIFDANLTTILTAIILYAVGTGPIQGFAITLSLGVAISMFTAITGTRALVNALYGGKQLKKLLI

SEQ ID NO: 121 - DNA, 291 bp, H. influenzae
  CDS (1)...(291): HI-0241
atggaagcacaaagcccaatgtccacgctatttattttcgtgatctttggtttaattttctactttatgatttatcgcccgcaagctaaacgcaataaagaacacaaaaaattgatgtctgagcttgcaaaaggtactgaagttttaaccgctggtggtgtaatcggcaaaattactaaagtaaccgaaggtagcgatagcatcgtgattgcgttaaacgacacgacagaaattacgattaatcgtaactacattgtgagcgttcttcctaaaggttcattaaaatcactt

SEQ ID NO: 122 - protein, 97 aa, H. influenzae
MEAQSPMSTLFIFVIFGLIFYFMIYRPQAKRNKEHKKLMSELAKGTEVLTAGGVIGKITKVTEGSDSIVIALNDTTEITINRNYIVSVLPKGSLKSL

SEQ ID NO: 123 - DNA, 219 bp, H. influenzae
  CDS (1)...(219): HI-0242
atgaaatatcagctcaatttaaccgcacttcgatgccccattcctcttttaagtgctaaaaaagccttaaaaaatttggataaaaatgatgagctaatgttgatcttaaaccttgaaagtgcggtggaaaatttttctatttttgccgaagaaaattctgttgctttggtcgagcaatattacgcttcggaaaaagaatttatcgttatcttgaaaaaa

SEQ ID NO: 124 - protein, 73 aa, H. influenzae
MKYQLNLTALRCPIPLLSAKKALKNLDKNDELMLILNLESAVENFSIFAEENSVALVEQYYASEKEFIVILKK

SEQ ID NO: 125 - DNA, 1443 bp, H. influenzae
  CDS (1)...(1443): HI-0183
atgattatggaatttgaattttcaaaaatgttagaagaagtgctaacttggatagtcgcacaccttgatggacctttatgggatgccaccattattattttgcttgggactggtctattttttaccattacaacaggatttgtgcagttccgtttattcccagcaagccttcgtgaaatgtggtttggtcgttcggtggaggggagttcattaacacctttccaagcgtttacaacaggtcttgcgagccgcgttggtgtgggtaacattggtggggttgcaacggcaatcgccttagggggcgaaggcgcagtgttttggatgtgggtaacggcatttattggtatgtcgagtgctttcgctgaatctacccttgctcaattatttaaaattcaagataaagatggatcattccgtggcggccctgcttattatattgtgcaaggtttaaaatcacgttgtatggcagtggcttttgcgcttgcattaatttttacatttggttttgcctttaattctgtgcaggcaaactctattgttgaagcaaccagcaatgcgtggaattggaaaggggaatatgtcggtatttcattagtaatttttaccgcacttattattttcggtggcgttaagcgtattgcgattatttcgagcaaccttgtaccaatgatggcacttttctatttgattatggcggtaattattcttggcatgcatattgatatgatcccttccgtgattcatcgtattgttcaaagtgcatttagttttgatgctgccgctggcggaatgtttggtgcattggtatcgaaagcaatgatgatggggattaaacgtggtttattctcaaacgaagcagggatgggatctgcgcctaattcggctgcagcagctcacgttaagcatccagttagccaaggtttagtgcaaatgctcggggtgtttgttgatacaatgatcgtttgtacttgtactgccgttattattttgctttcgaataattatggtagcgaaacgctcaaaagtatctctcttacgcaaaatgctttgcaataccacataggtgaatttggggcgcatttcctggcgtttatcttattgttattcgcttattcttctattattggtaactatgcttatgcggaaagcaacatccgttttatcaagaataaaccttggttggtcttgttgttccgtttaatggtgctatttttcgtgtatttcggtgcggttcgctctggtaatgtggtgtggaatttcgcagatacggtgatggctgtcatggcaatcattaacttgatcgcaattttgatgttgtcgccaatcgtatggaaattaatgaaagattatcaacgccagcttaaagaaggaaaaacgccagagtttaaaattgatgaataccctgaattacgtaagaaaatatttgattcccgcatttggaaa

SEQ ID NO: 126 - protein, 481 aa, H. influenzae
MIMEFEFSKMLEEVLTWIVAHLDGPLWDATIIILLGTGLFFTITTGFVQFRLFPASLREMWFGRSVEGSSLTPFQAFTTGLASRVGVGNIGGVATAIALGGEGAVFWMWVTAFIGMSSAFAESTLAQLFKIQDKDGSFRGGPAYYIVQGLKSRCMAVAFALALIFTFGFAFNSVQANSIVEATSNAWNWKGEYVGISLVIFTALIIFGGVKRIAIISSNLVPMMALFYLIMAVIILGMHIDMIPSVIHRIVQSAFSFDAAAGGMFGALVSKAMMMGIKRGLFSNEAGMGSAPNSAAAAHVKHPVSQGLVQMLGVFVDTMIVCTCTAVIILLSNNYGSETLKSISLTQNALQYHIGEFGAHFLAFILLLFAYSSIIGNYAYAESNIRFIKNKPWLVLLFRLMVLFFVYFGAVRSGNVVWNFADTVMAVMAIINLIAILMLSPIVWKLMKDYQRQLKEGKTPEFKIDEYPELRKKIFDSRIWK

SEQ ID NO: 127 - DNA, 1071 bp, H. influenzae
  CDS (1)...(1071): HI-0196
atggctggtaatacaattggacaacttttccgtgtgacaacctttggagagtcacatggtattgcattaggctgtatcgttgatggcgtgccaccaaatctcgaattatccgagaaagatattcagccagatttagatcgtcgtaaaccaggaacatctcgatatacgacgcctcgtcgtgaagatgacgaagttcaaattttatctggtgtgtttgaaggaaaaaccacaggcacaagtattgggatgatcattaaaaatggagatcagcgttcgcaagattatggtgacattaaagatcgtttccgcccaggtcatgcggattttacctatcagcaaaagtatggaatccgtgattatcgtggcggtgggcgttcgtcagcacgtgaaacagcgatgcgggttgctgcaggggctattgcgaaaaaatatttacgcgaacattttggcattgaggtgcgaggttttttaagccaaatcggtaatataaaaattgctccgcagaaagtgggacaaattgattgggaaaaggtaaacagtaatccattcttttgtcctgatgaaagtgcggtagaaaaattcgatgaattgatccgtgaacttaaaaaagaaggagattctattggcgcaaaacttactgttattgcagaaaatgtacctgtaggattgggcgagccagtatttgaccgtttagatgccgatcttgctcacgcattaatgggaattaatgcagtaaaaggtgtagaaattggcgatggctttgctgtggttgaacaacgaggttcggaacatcgtgatgaaatgacacctaatggctttgaaagtaatcatgcaggcggtattttaggcggaattagttcaggacaaccaattatcgccactattgcactaaaaccaacttcaagcattacgattcctggtcgttcaatcaatcttaatggtgaagccgtagaagttgtaacaaaaggtcgtcacgatccttgtgtggggattcgtgctgtgccaattgcggaagctatggtggcgattgtcttattagatcatctcttacgttttaaggcacagtgtaaa

SEQ ID NO: 128 - protein, 357 aa, H. influenzae
MAGNTIGQLFRVTTFGESHGIALGCIVDGVPPNLELSEKDIQPDLDRRKPGTSRYTTPRREDDEVQILSGVFEGKTTGTSIGMIIKNGDQRSQDYGDIKDRFRPGHADFTYQQKYGIRDYRGGGRSSARETAMRVAAGAIAKKYLREHFGIEVRGFLSQIGNIKIAPQKVGQIDWEKVNSNPFFCPDESAVEKFDELIRELKKEGDSIGAKLTVIAENVPVGLGEPVFDRLDADLAHALMGINAVKGVEIGDGFAVVEQRGSEHRDEMTPNGFESNHAGGILGGISSGQPIIATIALKPTSSITIPGRSINLNGEAVEVVTKGRHDPCVGIRAVPIAEAMVAIVLLDHLLRFKAQCK

SEQ ID NO: 129 - DNA, 975 bp, H. influenzae
  CDS (1)...(975): HI-0239
atgatgaaactttttacaaaagataaagacggacattttatccgtgaaatcaatgggataaagctcccgttcccattgactgaatttatgaaagtgcgtaaattgggttatatattatccgcacttttgatggtaatttctctattttttattattaccaaaggatttaactggggcttagattttactggtggagtggtatttgatactcacttctcgcagtccgctaaccttgaacaaattcgtagtaaacttcacgaaaatggaattgaaagcccaattgtacaaaccacaggatcggttcaggatgtgatgattcgtttacctgcaagtaataatgattctaccattggtgaacacgtcaaaagtatgctacagaatgtagataaagacattcaaattcgcagtattgagttcgttggcccaaatgttggtgaagaattagcacaaggtgcggtatatgcgactttagcgacattagcaatggtgcttatttatgtggggtcacgttttgaatggcgtttaggctttggcagtatcgcttctcttgcgcacgacgtcattattacgctaggggtattctctgcattacaaattgaaattgatcttacttttgtcgcagcgattttatctgtggtgggttactccatcaacgatagtattgtggtatttgaccgggttcgtgaaaatttccgaaaaattagacgattggatacgattgatattattgatatttctttaacgcaaactttatcaagaactatcattacttcggttactacattagttgtcgtgatggcattgttcttctttggtggtccttccattcataacttttcacttgctttactcgtaggtattggatttggtacttattcctcgatttttgttgccattgccattgcatacgatgttggtttacgtcgtgaacatatgatcccacctaaagtagataaagaaattgatgaattacct

SEQ ID NO: 130 - protein, 325 aa, H. influenzae
MMKLFTKDKDGHFIREINGIKLPFPLTEFMKVRKLGYILSALLMVISLFFIITKGFNWGLDFTGGVVFDTHFSQSANLEQIRSKLHENGIESPIVQTTGSVQDVMIRLPASNNDSTIGEHVKSMLQNVDKDIQIRSIEFVGPNVGEELAQGAVYATLATLAMVLIYVGSRFEWRLGFGSIASLAHDVIITLGVFSALQIEIDLTFVAAILSVVGYSINDSIVVFDRVRENFRKIRRLDTIDIIDISLTQTLSRTIITSVTTLVVVMALFFFGGPSIHNFSLALLVGIGFGTYSSIFVAIAIAYDVGLRREHMIPPKVDKEIDELP

SEQ ID NO: 131 - DNA, 36 bp, Artificial Sequence
  Primer AT-Cm (+) containing XmnI restriction sites
attaatgaacatgttctacctgtgacggaagatcac

SEQ ID NO: 132 - DNA, 36 bp, Artificial Sequence
  Primer AT-Cm (-) containing XmnI restriction sites
attaatgaacatgttcaccgggtcgaatttgctttc

SEQ ID NO: 133 - DNA, 28 bp, Artificial Sequence
  Primer specific for AT-Cm 542
aaagaaaaataagcacaagttttatccg

SEQ ID NO: 134 - DNA, 28 bp, Artificial Sequence
  Primer specific for metE 5′
atgacaacatcacatattttaggctttc

SEQ ID NO: 135 - DNA, 22 bp, Artificial Sequence
  Primer specific for metE 3′
cgctaattccgcacgtaatttt

SEQ ID NO: 136 - DNA, 21 bp, Artificial Sequence
  Primer AT-Cm Seq (+)
attggtgcccttaaacgcctg

SEQ ID NO: 137 - DNA, 21 bp, Artificial Sequence
  PrimerAt-Cm Seq (-)
ttacgtgccgatcaacgtctc

(19) United States
(12) Patent Application Publication
(10) Pub. No.: US 2003/0021813 A1
(43) Pub. Date: Jan. 30, 2003

(54) Essential bacteria genes and genome scanning in Haemophilus influenzae for the identification of 'essential genes'

(76) Inventors: Linda E. Chovan, Kenosha, WI (US); Paul E. Hessler, Hainesville, IL (US); Karl A. Reich, Libertyville, IL (US)

Correspondence Address: STEVEN F. WEINSTOCK, ABBOTT LABORATORIES, D-377 - AP6A-1, 100 ABBOTT PARK ROAD, ABBOTT PARK, IL, 60064, US

(21) Appl. No.: 10/260,877

(22) Filed: Sep. 30, 2002

Related U.S. Application Data
(62) Division of application No. 09/649,145, filed on Aug. 25, 2000.
Application No. 09/649,145 is a continuation-in-part of application No. 09/368,382, filed on Aug. 4, 1999, now abandoned.

(51) Int. Cl. A61K 39/102; C12Q 1/68; C07H 21/04; C12N 9/00; C12P 21/02; C12N 1/21; C12N 15/74

(52) U.S. Cl. 424/256.1; 435/6; 435/320.1; 435/252.3; 435/183; 435/69.3; 536/23.7

(57) ABSTRACT

Essential bacteria genes and a method for identifying ‘essential genes’ (i.e., genes which are essential to a bacterium's survival) using an in vitro transposition system, a small (975 bp) insertional element containing an antibiotic resistance cassette and mapping these inserts relative to the deduced open reading frames of H. influenzae by PCR and Southern analysis.

### CROSS-REFERENCE TO RELATED APPLICATION

This application is a continuation-in-part of U.S. application Ser. No. 09/368,382 filed Aug. 4, 1999, from which priority is claimed pursuant to 35 U.S.C. §120 and which is incorporated herein by reference in its entirety.

### FIELD OF THE INVENTION

This invention relates to newly identified polynucleotides, polypeptides, and their production, methods and uses, as well as variances, isolated from Haemophilus influenzae, the polynucleotide sequences of which are required for survival.

### BACKGROUND

Haemophilus influenzae is Gram negative human pathogen. It is responsible for both invasive and non-invasive disease in both children and adults. The usual infections include middle ear (otitis media) and upper respiratory tract infections. There is an effective pediatric vaccine that has reduced the incidence of invasive disease in children (at least in the first world where the vaccine is given) but this has led to no decrease in adult disease—probably because the organism is normal resident of the human naso-pharyxn.

Haemophilus influenzae, often referred to a H. flu for convenience, is a family of bacteria all of which can cause diseases in people. (The bacteria does not have anything to do with influenza, but when first identified it was thought to cause flu, hence the name.) There are six sero types of H. flu known; most H flu-related disease is caused by type B, or “HIB”.

Until a vaccine for HIB was developed, HIB was one of the two most common causes of otitis media, sinus infections, and bronchitis. More important, HIB was also the most common cause of meningitis, and a frequent culprit in cases of pneumonia, septic arthritis (joint infections), cellulitis (infections of soft tissues), and pericarditis (infections of the membrane surrounding the heart). One of the most dangerous results of HIB infection was epiglottis, an infection of the “flap” at the top of the windpipe that could kill a child by blocking air to the lungs.

Before the vaccine was introduced, there were about 20,000 serious cases of HIB infections in the United States every year, most of which were of meningitis. Since the vaccine became required, that number has dropped to about one-sixth to one-eighth of what it was. Currently, approximately 12,000 cases of HIB infections are reported each year in the United States. Although, the mortality rate is less than 10 percent, 10 to 15 percent of the survivors are left with neurological complications. Meningitis caused by H. influenzae has a seasonal distribution, with major incidences of the disease occurring in the fall and spring.

The vaccine is given 2-3 times in the first 6 months of life after birth, as a newborn, followed by a single dose at age 12-18 months. (There are two different HIB vaccines available; they are both very effective, but the dosage schedule differ between the two types.)

In children under the age of 5, haemophilus influenzae type B (Hib) is one of the leading causes of invasive bacterial infection. It is the leading cause of meningitis* in this age group, killing 5 percent of infected children even when antibiotics are used to fight the disease.

The infection strikes most frequently between 6 and 12 months of age; 75 percent of all cases occur before 24 months of age. Certain groups—including African-Americans, Hispanics, Native American Indians and children who attend day-care centers—are at a higher risk of infection.

Even though HIB infections in adults are rare, they occur more frequently when the patient is compromised by respiratory problems, diabetes, AIDS, or alcoholism. Infection is manifested as pneumonia.

The first symptoms of haemophilus influenzae infection often resemble those of a cold, with a fever and headache. However, when the iinfection reaches the covering of the brain (miningitis), nausea, vomiting and seizures may occur, making this a serious medical emergency.

Haemohhilus species make up a substantial portion of the indigenous microflora of the upper respiratory tract. Nearly all individuals over the age of 1 year are carriers for one or more species of Haemophilus. Species found in the upper respiratory tract include H. influenzae, H. parainfluenzae, H. haemolyticus, and H. paraphaemolyticus. Of these species H. influenzae is the most pathogenic.

H. influenzae is fastidious in its growth requirements. It grows best on chocolate agar or enriched media supplemented with two nutritional factors called X (hemin) and V (nicotinamide-adenine dinucleotide [NAD]). Colonies of H. influenzae increase in size if they are cultivated in the vicinity of other bacterial colonies, staphylococci, for example. This cooeprative effect is called the satellite phenomenon and is due to the production of NAD by the staphylococcal colonies.

H. influenzae can be divided into two groups: encapsulated and nonencaptsulated.

Infection with H. influenzae occurs following inhalation of respiratory droplets from patients or carriers. Most invasive infections in the upper respiratory tract are caused by type b encapsultated strains (HIB). HIB serotypes are associated primarily with systemic infections that are the result of invasion of the bloodstream, for example, meningitis, epiglottitis, cellulitis, septic arthritis, and pneumonia. Type b serotypes are the principal cause of bacterial meningitis in children under 4 years old. In this group of children, meningitis, even after chemotherapy, can lead to serious sequelae such as mental retardation.

The mechanism of pathogenesis of type b serotypes is not fully understood. Adnerence of bacteria to the respiratory tract may be due to delayed mucociliary clearance. For example, smoking or prior viral infection could cause loss of ciliary epithelium. As the epithelial surface becomes damaged, host receptors could be exposed, leading to interaction with bacterial adhesins. Bacteria invade the bloodstream, where they multiply and cross the blood-brain barrier. The capsule of HIB organisms appears to protect them from intravascular clearance mechanisms. Bacterial products as well as cell wall lipopolysaccharide (LPS) and peptidoglycan may play a role in the inflammation and tissue damage associated with meningitis.

H. influenzae is found in the upper respiratory tract of most healthy individuals. HIB serotypes are found in the upper respiratory tract of less than 1 percent of children 6 months or younger. In infants 2 months or younger, maternal antibody provides protection from infection by HIB strains. The majority of HIB meningitis infections occur in chidlren between the ages of 2 and 18 months. From the ages of 2 months to 5 years, HIB serotypes can be found in 5 percent of the children. Most children over the age of 5 years and adults will have naturally acquired immunity to HIB serotypes. Consequently 95 percent of HIB disease is found in children less than 4 years old. Nonencapsulated strains become less prevalent as commensals with increasing age.

Seventy-five percent of the H. influenzae strains in the upper respiratory tract are nonencapsulated. Nonencapsulated strains rarely cause systemic disease. Mucous membrane infections such as otitis media, sinusitis, bronchitis, alveolitis, conjunctivitis, and infections involving the female genital tract during parturition, however, common. Pneumonia caused by nonencapsulated strains is more common in the elderly and in patients with chronic bronchitis. These strains (along with Streptococcus pneumoniae) are the most frequent cause of otitis media in children between the ages of 6 and 24 months. By the age of 3 years, more than two-thirds of children have had one or more episodes of acute otitis media. Meningitis occurs primarily in predisposed patients. Of all meningitis cases in adults, about 50 percent are caused by nonencapsulated strain.

Ampicillin and chloramphenicol were once considered the most effective drugs in treating infections caused by H. influenzae, but drug-resistant strains have now become prevalent and as a result, sensitivity tests must be performed on clinical isolates before an antimicrobial regimen is begun.

The increasing incidence of antibiotic resistant bacteria in clinical practice has stimulated renewed interest within the pharmaceutical industry in searching for, and developing, new ways to combat H. influenzae infections. One approach used in this work is identifying and targeting genes essential to this bacteria's survival. Until recently, the identification of appropriate bacterial essential genes has been a slow, laborious process and limited to a few well-defined bacterial functions. Present inventors have found a number of bacterial sequences which are key to the bacterial's growth and survival.

Specifically, the inventors have analyzed genomic sequence from H. influenzae bacterial pathogens and revealed a large fraction of open reading frames (ORFs) of unknown or hypothetical function, which are required for bacterial growth and survival. These genes can be utilized to identify potential anti-bacterial compounds. Accordingly, an experimental method to ‘annotate’ a bacterial genome at a simple level has been developed in order to deduce the ORF required for growth under the chosen conditions. This would be one criterion for choosing an anti-bacterial target for development and for use to screen compounds which affect this target.

### SUMMARY OF THE INVENTION

This invention relates to essential bacterial genes are necessary for the bacterium's growth and survival, which could serve as potential anti-bacterial targets.

Another aspect of the invention relates to a method for the identification of essential genes. Two methods are contemplated: ‘mutation exclusion’ or ‘zero-time analysis’. Mutation exclusion consists of growing an insertional library and identifying open reading frames that do not contain insertional elements: in a growing population of bacteria, insertions in essential genes are excluded. Zero-time analysis consists of following the fate of individual insertions after transformation in a growing culture: the loss of inserts in essential genes are followed over time. Both methods of analysis permit the identification of genes required for bacterial survival.

Specifically, once a mutant organism (strain) is identified, routine techniques may be used for transformation, amplification, isolation, purification, and sequencing the gene carrying the mutation. Essential survival genes are required for growth (e.g., metabolism, division, or reproduction). Such genes and gene products are useful in developing therapeutic agents such as antifungal, antibacterial, and antiparasitic agents; insecticidal agents; and preventive antimicrobial agents. Therapeutic agents can reduce or prevent growth, or decrease pathogenicity or virulence, and preferably, kill the organism. The genes and gene products identified by the invention can also be used to develop antimicrobial agents which are effective in preventing microbial infection, e.g., agents which are useful in the treatment of an established infection.

Therapeutic agents can be developed from the identification of essential genes of organisms such as bacteria or fungi. Preferably, a gene product (e.g., a protein or an RNA molecule) identified by the methods disclosed herein is distinct from the gene products targeted by existing drugs such as antibiotic or antifingal agents. The disclosed gene selection methods establish that the gene product is essential for survival of the organism. Such an identified gene product therefore serves as a novel target for therapeutics based on a mechanism which is likely distinct from the mechanisms of existing drugs. Similarly, distinct from known compounds is a compound which inhibits the function of a gene product identified by methods disclosed herein, for example, by producing a phenotype or morphology similar to that found in the original mutant strain.

Details of the essential genes identified, the mutant library construction, the mapping strategy and examples of mutant exclusion and zero-time analysis are detailed below.

### BRIEF DESCRIPTION OF THE DRAWINGS

FIG. 1. Features and Partial Restriction Maps of in vitro Transposition Cassettes. Relevant restriction sites, positions of start and stop codons and position of open reading frame coding for antibiotic resistance determinants are indicated. Solid bars indicate position of U3 terminii recognized by Ty-1 transposase. Upper diagram: AT-2, lower diagram: AT-Cm. Position of AT-Cm specific insert anchored primer is indicated by the half arrow.

FIG. 2. Southern Analysis of Antibiotic Resistant H. influenzae Isolates. Panel A: Genomic Southern of trimethoprim resistant colonies. Panel B: Genomic Southern of chloroamphenicol resistant colonies. Lanes 1-24, 1 colony/lane, Lanes 25-30, three colonies/lane. Panel A, lanes 1-31, EcoRI digest; lanes 31-36 EcoRI/BamHI double digest. Panel B, lanes 1-36, EcoRI digest. Lane +: positive controls for Southern hybridization using AT-2 and AT-Cm, respectively.

FIG. 3. Detection of metE Insert Mutant by PCR and Southern Analysis. Southern blot of dilutions of metE mutant DNA with genomic DNA from small insert library. Positions of known metE insert and library mutants are shown. Genome equivalents indicate the calculated copies of PCR template in the reactions. Schematic shows position of the PCR primers relative to metE coding region and AT-Cm insert.

FIG. 4. ‘Zero time’ Analysis of metE Insertion Loss. Aliquots from growing cultures were removed at the indicated times and processed for PCR and Southern analysis (see text). Results from minimal media with (upper panel) and without (lower panel) methionine. The optical density of bacterial cultures (right hand panel) for mimimal media with (solid line) and without (dashed line) methionine are shown. Schematic illustrates the position of PCR primers used in the analysis.

FIG. 5. ‘Mutation Exclusion analysis’ of HI#991-998. Ethidium stained agarose gel and Southern analysis of insert anchored PCR reactions using primers specific for HI#991-998 (lanes 2-9)(see text for details). ORF map of chromosomal region; arrows indicate direction of transcription and relative sizes of open reading frames. The position and orientation of ORF specific primers are shown by the half arrows. The deduced location of inserts are indicated by the vertical bars above the ORF map.

### DETAILED DESCRIPTION

The minimum number of genes/functions required for autonomous bacterial growth has been variously estimated. While it is clear that bacteria possess redundant, or back-up functions, there are individual genes that are absolutely required for growth or viability.

The data supplied is experimental, as opposed to computational, method for identifying essential genes in Haemophilus influenzae. The technique makes use of in vitro transposition to generate a large, random, insertional mutant library and a combination of PCR and Southern analysis to map the chromosomal location of the inserts. The choice of H. influenzae was influenced by the quality of its genomic sequence, the ease and efficiency of DNA transformation in this organism and its continued importance as a human pathogen. The details of the library construction, the insert mapping strategy and the analysis used for identifying previously unknown essential genes are described.

Glossary of Terms

Unless otherwise stated, the following terms shall have the following meanings:

“Essential genes” are defined as genes, which, if they loose their function via mutation or some other occurance, will cause the death of a bacterium. In other words, a mutation in an essential gene results in bacterial death either immediately or over several generations.

A polynucleotide “derived from” or “specific for” a designated sequence refers to a polynucleotide sequence that comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The sequence may be complementary or identical to a sequence that is unique to a particular polynucleotide sequence as determined by techniques known in the art. Comparisons to sequences in databanks, for example, can be used as a method to determine the uniqueness of a designated sequence. Regions from which sequences may be derived, include but are not limited to, regions encoding specific epitopes, as well as non-translated and/or non-transcribed regions.

The derived polynucleotide will not necessarily be derived physically from the nucleotide sequence of interest under study, but may be generated in any manner, including, but not limited to, chemical synthesis, replication, reverse transcription or transcription, that is based on the information provided by the sequence of bases in the region(s) from which the polynucleotide is derived. As such, it may represent either a sense or an antisense orientation of the original polynucleotide. In addition, combinations of regions corresponding to that of the designated sequence may be modified in ways known in the art to be consistent with the intended use.

A “fragment” of a specified polynucleotide refers to a polynucleotide sequence that comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the specified nucleotide sequence.

The term “primer” denotes a specific oligonucleotide sequence that is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., PNA as defined hereinbelow) which can be used to identify a specific polynucleotide present in samples bearing the complementary sequence.

“Encoded by” refers to a nucleic acid sequence that codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence. Thus, a “polypeptide,” “protein,” or “amino acid” sequence has at least about 50% identity, preferably about 60% identity, more preferably about 75-85% identity, and most preferably about 90-95% or more identity with a BS325 amino acid sequence. Further, the BS325 “polypeptide,” “protein,” or “amino acid” sequence may have at least about 60% similarity, preferably at least about 75% similarity, more preferably about 85% similarity, and most preferably about 95% or more similarity to a polypeptide or amino acid sequence of the present invention.

A “recombinant polypeptide,” “recombinant protein,” or “a polypeptide produced by recombinant techniques,” which terms may be used interchangeably herein, describes a polypeptide that by virtue of its origin or manipulation is not associated with all or a portion of the polypeptide with which it is associated in nature and/or is linked to a polypeptide other than that to which it is linked in nature. A recombinant or encoded polypeptide or protein is not necessarily translated from a designated nucleic acid sequence. It also may be generated in any manner, including chemical synthesis or expression of a recombinant expression system.

The term “synthetic peptide” as used herein means a polymeric form of amino acids of any length, which may be chemically synthesized by methods well known to the routineer. These synthetic peptides are useful in various applications.

The term “polynucleotide” as used herein means a polymeric form of nucleotides of any length, either ribonucleotides or deoxyribonucleotides. This term refers only to the primary structure of the molecule. Thus, the term includes double- and single-stranded DNA, as well as double- and single-stranded RNA. It also includes modifications, such as methylation or capping and unmodified forms of the polynucleotide. The terms “polynucleotide,” “oligomer,” “oligonucleotide,” and “oligo” are used interchangeably herein.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively. Two or more polynucleotide sequences can be compared by determining their “percent identity.”Two or more amino acid sequences likewise can be compared by determining their “percent identity.” The percent identity of two sequences, whether nucleic acid or peptide sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An implementation of this algorithm for nucleic acid and peptide sequences is provided by the Genetics Computer Group (Madison, Wis.) in their BestFit utility application. The default parameters for this method are described in the Wisconsin Sequence Analysis Package Program Manual, Version 8 (1995) for example the GAP program (available from Genetics Computer Group, Madison, Wis.). Other equally suitable programs for calculating the percent identity or similarity between sequences are generally known in the art.

“Purified polynucleotide” refers to a polynucleotide of interest or fragment thereof that is essentially free, e.g., contains less than about 50%, preferably less than about 70%, and more preferably less than about 90%, of the protein with which the polynucleotide is naturally associated. Techniques for purifying polynucleotides of interest are well known in the art and include, for example, disruption of the cell containing the polynucleotide with a chaotropic agent and separation of the polynucleotide(s) and proteins by ion-exchange chromatography, affinity chromatography and sedimentation according to density.

“Purified polypeptide” or “purified protein” means a polypeptide of interest or fragment thereof that is essentially free of, e.g., contains less than about 50%, preferably less than about 70%, and more preferably less than about 90%, cellular components with which the polypeptide of interest is naturally associated. Methods for purifying polypeptides of interest are known in the art.

The term “isolated” means that the material is removed from its original environment (e.g., the natural environment if it is naturally occurring). For example, a naturally occurring polynucleotide or polypeptide present in a living animal is not isolated, but the same polynucleotide or DNA or polypeptide, that is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

“Polypeptide” and “protein” are used interchangeably herein and indicate at least one molecular chain of amino acids linked through covalent and/or non-covalent bonds. The terms do not refer to a specific length of the product. Thus peptides, oligopeptides and proteins are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

A “fragment” of a specified polypeptide refers to an amino acid sequence which comprises at least about 3-5 amino acids, more preferably at least about 8-10 amino acids, and even more preferably at least about 15-20 amino acids derived from the specified polypeptide.

“Recombinant host cells,” “host cells,” “cells,” “cell lines,” “cell cultures,” and other such terms denoting microorganisms or higher eukaryotic cell lines cultured as unicellular entities refer to cells that can be, or have been, used as recipients for recombinant vector or other transferred DNA, and include the original progeny of the original cell that has been transfected.

As used herein “replicon” means any genetic element, such as a plasmid, a chromosome or a virus, that behaves as an autonomous unit of polynucleotide replication within a cell.

A “vector” is a replicon in which another polynucleotide segment is attached, such as to bring about the replication and/or expression of the attached segment.

The term “control sequence” refers to a polynucleotide sequence that is necessary to effect the expression of a coding sequence to which it is ligated. The nature of such control sequences differs depending upon the host organism. In prokaryotes, such control sequences generally include a promoter, a ribosomal binding site and terminators; in eukaryotes, such control sequences generally include promoters, terminators and, in some instances, enhancers. The term “control sequence” thus is intended to include at a minimum all components whose presence is necessary for expression, and also may include additional components whose presence is advantageous, for example, leader sequences.

“Operably linked” refers to a situation wherein the components described are in a relationship permitting them to function in their intended manner. Thus, for example, a control sequence “operably linked” to a coding sequence is ligated in such a manner that expression of the coding sequence is achieved under conditions compatible with the control sequence.

The term “open reading frame” or “ORF” refers to a region of a polynucleotide sequence that encodes a polypeptide. This region may represent a portion of a coding sequence or a total coding sequence.

A “coding sequence” is a polynucleotide sequence that is transcribed into mRNA and translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to, mRNA, cDNA and recombinant polynucleotide sequences.

The term “transfection” refers to the introduction of an exogenous polynucleotide into a prokaryotic or eucaryotic host cell, irrespective of the method used for the introduction. The term “transfection” refers to both stable and transient introduction of the polynucleotide, and encompasses direct uptake of polynucleotides, transformation, transduction, and f-mating. Once introduced into the host cell, the exogenous polynucleotide may be maintained as a non-integrated replicon, for example, a plasmid, or alternatively, may be integrated into the host genome.

The term “individual” as used herein refers to vertebrates, particularly members of the mammalian species and includes, but is not limited to, domestic animals, sports animals, primates and humans; more particularly, the term refers to humans.

The term “sense strand” or “plus strand” (or “+”) as used herein denotes a nucleic acid that contains the sequence that encodes the polypeptide. The term “antisense strand” or “minus strand” (or “−”) denotes a nucleic acid that contains a sequence that is complementary to that of the “plus” strand.

“Purified product” refers to a preparation of the product that has been isolated from the cellular constituents with which the product is normally associated and from other types of cells that may be present in the sample of interest.

“PNA” denotes a “peptide nucleic acid analog” that may be utilized in a procedure such as an assay described herein to determine the presence of a target. “MA” denotes a “morpholino analog” that may be utilized in a procedure such as an assay described herein to determine the presence of a target. See, for example, U.S. Pat. No. 5,378,841, that is incorporated herein by reference. PNAs are neutrally charged moieties that can be directed against RNA targets or DNA. PNA probes used in assays in place of, for example, the DNA probes of the present invention, offer advantages not achievable when DNA probes are used. These advantages include manufacturability, large scale labeling, reproducibility, stability, insensitivity to changes in ionic strength and resistance to enzymatic degradation that is present in methods utilizing DNA or RNA. These PNAs can be labeled with (“attached to”) such signal generating compounds as fluorescein, radionucleotides, chemiluminescent compounds and the like. PNAs or other nucleic acid analogs such as MAs thus can be used in assay methods in place of DNA or RNA. Although assays are described herein utilizing DNA probes, it is within the scope of the routineer that PNAs or MAs can be substituted for RNA or DNA with appropriate changes if and as needed in assay reagents.

### EXAMPLE 1

Strain construction. Haemophilus influenzae strain BC200 (the kind gift of Jane Setlow) was cured of plasmid pDM2 by growth in brain heart infusion supplemented with NAD (10 μg/mL) and hemin (12 μg/mL) (sBHI) at 37° C. without antibiotics. After serial passage, individual isolates were tested for sensitivity to ampicillin and chloroamphenicol. A sensitive isolate was examined for plasmid content and transformation efficiency and designated NP200 (for No Plasmid).

Competent Cell Preparation. NP200 competent cells were prepared using competence-inducing MIV medium (4). Cells were stored at −80° C. in 1.0 mL aliquots.

Transformation of NP200 Competent Cells. Frozen competent cells were thawed on wet ice, spun briefly and re-suspended in 1.0 ml of freshly prepared MIV medium (4). One microgram of DNA was added and the cells incubated at 37° C. for 30 mins. Fresh sBHI was then added (5 ml) and the cells incubated for an additional 90 mins (with shaking). Chloramphenicol was added to a final concentration of 1.5 μg/mL and the cells for grown for an additional 90 mins. The culture was then plated on sBHI-agar containing 1.5 μg/ml chloroamphenicol.

Genomic DNA preparation. The CTAB method (3) was used for the isolation of genomic DNA from H. influenzae with the addition of 10 μl of RNase A (50 μg/ml) and incubation at 37° C. for 15 mins, prior to the second phenol extraction.

DNA Quantification. DNA was quantified fluorometrically (Turner Designs) relative to lambda standards using Pico green (Molecular Probes).

Generation of AT-Cm. The region from bp 19 to bp 3757 from pACYC184 (New England Biolabs) was PCR amplified using primers containing XmnI restriction sites (AT-Cm (+) ATTAATGAACATGTTCTACCTGTGACGGAAGATCAC; AT-Cm (−) ATTAATGAACATGTTCACCGGGTCGAATTTGCTTTC). The PCR product was purified by phenol/chloroform extraction, precipitated with NaOAc, and repeated ultrafiltration (Ultrafree CL, Millipore). The recognition sites for Ty-1 transposase (sequence in bold type) were generated by XmnI digestion of the purified DNA (XmnI sites are underlined).

In vitro transposition. Primer island transposition kits (Perkin Elmer) were used essentially as outlined by the manufacturer. Briefly, 1 μg of H. influenzae genomic DNA was mixed with transposase buffer, 0.2 μg of AT-Cm and 3 μl of transposase, in a final volume of 30 μl, for 3 hr at 30° C. The reaction was terminated by the addition of proteinase K and EDTA. The DNA was precipitated with ammonium acetate and single stranded gaps, introduced by the in vitro insertion reaction, were subsequently repaired.

DNA Repair Reaction. in vitro mutagenized genomic DNA was repaired with 2.5 μl of E. coli PolI (NEB), 1 l T4 DNA ligase (NEB), 20 mM dNTPs in 1× ligase buffer for 30 mins at 37° C. The DNA was precipitated with sodium acetate, washed carefully in 70% EtOH and stored at −20° C.

Mutant Library Construction. in vitro mutagenized genomic DNA was transformed into H. influenzae NP200 and the transformation mix plated on sBHI-agar containing 1.5 μg/mL chloroamphenicol. After 24 hrs, chloroamphenicol resistant colonies were pooled by the addition of sBHI (5 mL) to the plates and gently scraping the colonies together. The number of plates that were pooled determined the size of the mutant library. We routinely obtained 1000-3000 mutants from a single Ty-l reaction.

PCR reactions. TaKaRa taq polymerase was used according to the manufacturer in 50 μl reactions with 50 ng of genomic DNA as template. A three step PCR reaction was used: 94° C. (5 min)[1 cycle]; 94° C. (1 min), 62° C. (30 sec), 68° C. (2.5 min)[35 cycles]; 68° C. (10 min)[1 cycle].

Southern Analysis. Large format (25×20 cm) agarose gels were soaked sequentially with 0.1 N HCl and 0.4 M NaOH and transferred to Hybond N+ (Amersham) by vacuum blotting (BioRad). Membranes were prehybrized for 1 hr and hybridized overnight in 20 ml of hybridization solution (GIBCO) with P³³dCTP random-labeled probes (19). Membranes were washed twice in 2×SSC (42° C.) followed by two washes in 0.1×SSC (63° C.), exposed overnight to a phosphor screen and visualized by phosphoimaging (Molecular Dynamics).

Molecular weight markers. Four molecular weight markers (542 bp, 975 bp, 2151 bp and 4244 bp) that hybridize with an AT-Cm probe were constructed as follows: the 542 bp fragment was PCR amplified from AT-Cm using a primer pair consisting of primer AT-Cm (+) and primer AT-Cm 542; the 975 bp marker was XmnI digested AT-Cm; the 2151 bp fragment was ScaI/EcoRV digested pACYC184 and the 4244 bp marker was linearized pACYC184.

Oligonucleotides. PCR primers specific for At-Cm and mete (AT-Cm 542 AAAGAAAAATAAGCACAAGTTTTATCCG) were designed using OLIGO (MBInsights) with a calculated Tm of 70° C. (mete 5′-ATGACAACATCACATATTTTAGGCTTTC; metE 3′-CGCTAATTCCGCACGTAATTTT).

Genomic sequencing. H. influenzae genomic DNA (3-5 μg) was used as a template for PCR cycle sequencing (Perkin Elmer) using oligonucleotide primers AT-Cm Seq (+) ATTGGTGCCCTTAAACGCCTG and AT-Cm Seq (−) TTACGTGCCGATCAACGTCTC.

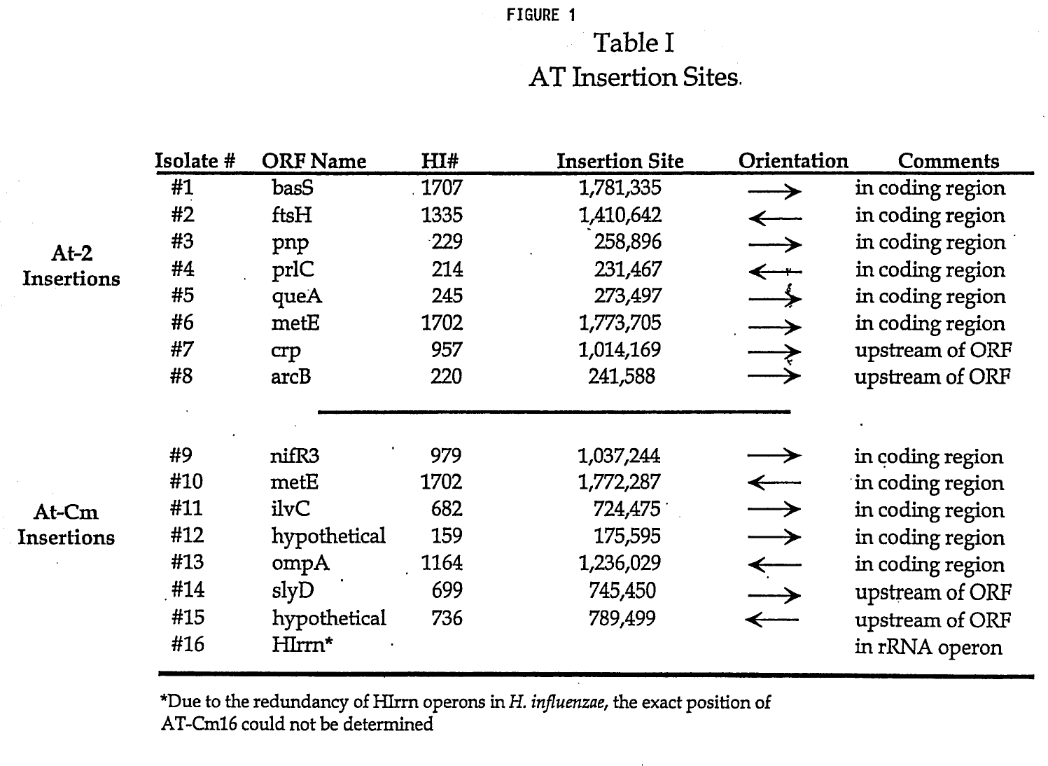

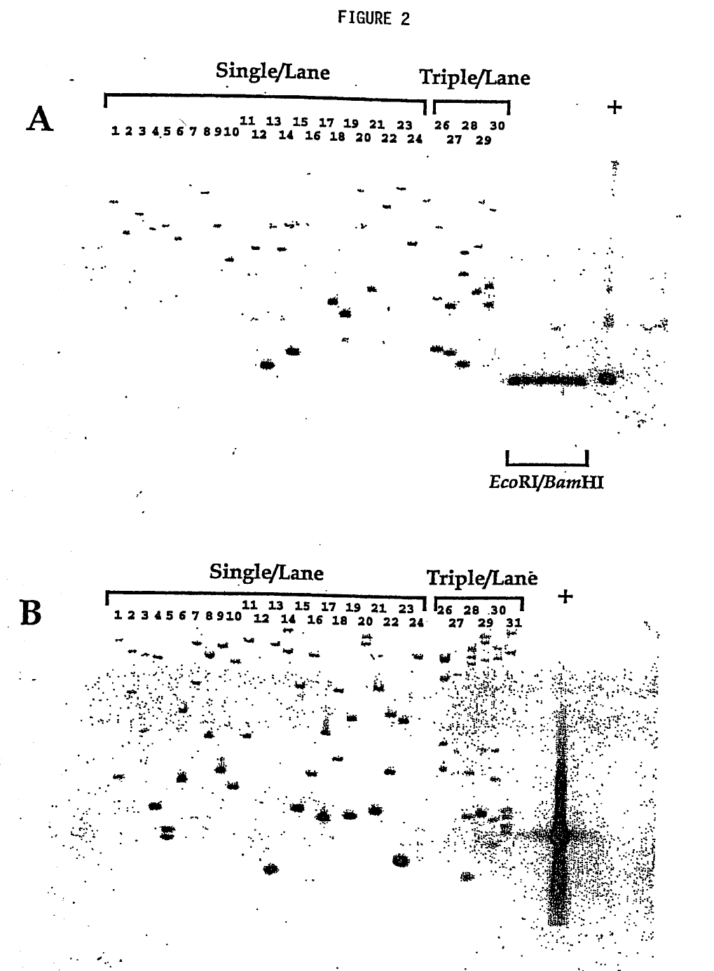

Characterization of in vitro transposon mutagenized H. influenzae. The in vitro transposition reaction catalyzed by Ty-1 randomly inserts a DNA fragment with defined ends into a DNA target (Devine, Scott E. and J. D. Boeke, “Efficient integration of artificial transposons into plasmid targets in vitro: a useful tool for DNA mapping, sequencing and genetic analysis”, Nucleic Acids Res. 22:3765-3772 (1994); and Braiterman, L. T., et al., “In frame linker insertion mutagenesis of yeast transposon Ty 1:phenotypic analysis”, Gene, 139:19-26 (1994)). This system was tested with two antibiotic resistance cassettes (FIG. 1) and high molecular weight H. influenzae genomic DNA as target. After in vitro reaction and repair (see Methods) the DNA was transformed into competent H. influenzae and the transformation mix plated on selective media (trimethoprim for AT-2 and chloroamphenicol for AT-Cm). The resultant antibiotic resistant colonies for the number and randomness of insertions into the H. influenzae chromosome were examined by Southern analysis (FIG. 2). Genomic DNA from overnight cultures inoculated from single colonies or three independently picked colonies was isolated, digested with EcoRI (FIG. 2, panel A and B, lanes 1-23) or with EcoRI/BamHI (FIG. 2, panel A, lanes 31-36), separated by agarose gel electrophoresis and transferred to nylon membranes. These filters were probed with a random primed ³³P-labelled AT-2 (FIG. 2, panel A) or AT-Cm (FIG. 2, panel B) probe. The single Southern-hybridizing band seen in each lane with the AT-2 probe is evidence that resistant clones contain a single AT-2 insertion (FIG. 2, panel A, lanes 1-23). The size distribution of Southern hybridizing genomic EcoRI fragments were interpreted as evidence for the randomness of insertion sites in the H. influenzae chromosome. The fidelity and integrity of the in vitro reaction was examined by digesting genomic DNA samples with restriction sites that are at each end of the AT-2 cassette (EcoRI/BamHI): the entire AT-2 insert should be released from high molecular weight DNA. A Southern hybridizing band can clearly be seen that migrates with the same apparent molecular weight as authentic AT-2 (FIG. 2, panel A, lanes 30-35) confirming that the in vitro reaction, transformation and selection proceeds such that an entire antibiotic cassette is randomly inserted into high molecular weight DNA.

A similar analysis was performed on chloroamphenicol resistant clones (FIG. 2, panel B). The AT-Cm cassette contains a unique internal EcoRI site (FIG. 1), therefore a single insertion will yield two Southern hybridizing bands when an EcoRI digested genomic Southern is probed with a randomly primed ³³P-labelled AT-Cm. The observed pattern was interpreted to indicate that for the AT-Cm cassette, insertions are also randomly distributed in the H. influenzae chromosome. The results from the multiple isolate cultures (FIG. 2, panels A and B, lanes 25-30) provide further evidence for the random nature of the insertion reaction and for the conclusion that each isolate contains a single insert: the number of observed bands can be accounted for by the number of colonies picked to grow the culture (1 band/colony for AT-2; 2 bands/colony for AT-Cm).

Identification of insertion sites. More precise localization of inserts in the H. influenzae chromosome was determined by direct sequencing. Oligonucleotide primers specific for either AT-2 or AT-Cm were designed (˜150 bp from the ends of the inserts, see Methods) that permitted the junctions between the cassettes and the H. influenzae genome to be identified by comparing our sequencing results to the H. influenzae genomic sequence (Fleischmann, R. D., M. D. et al., “Whole-genome random sequencing and assembly of Haemophilus influenzae Rd.”, Science, 269(5223):496-512, (1995)). The DNA template for the sequencing reactions was the genomic DNA used for Southern analysis (see above). The results (Table 1) show that the in vitro reaction can insert AT-2 and AT-Cm into a variety of DNA elements: open reading frames, intergenic regions and ribosomal operons. No sequence preferences for insertion sites were observed. Comparison of the sequence data derived from the outward reading primers (appropriate to each cassette) with the published H. influenzae genome, revealed no deletions or insertions near the transposon insertion sites. We interpret these results as further evidence that the in vitro reaction, repair and subsequent transformation, introduces no local DNA rearrangements or deletions near the insertion site. One isolate, AT-Cm10, contained an AT-Cm insert in mete (codon 603) and a strain bearing this mutation was reconstructed from isolated genomic DNA using standard techniques (see Methods).

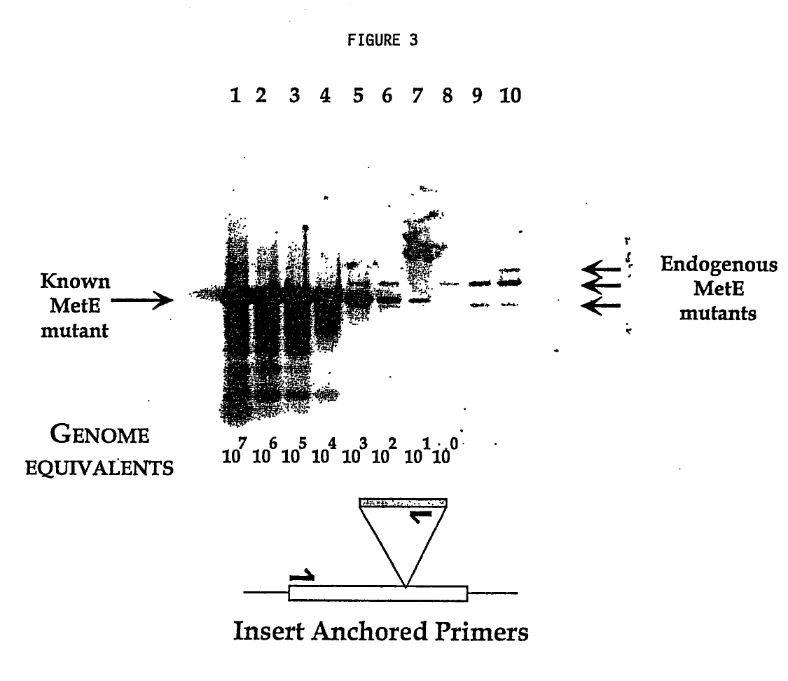

PCR and Southern Detection of Chromosomal Insertions. The strategy for identifying essential genes uses a technique for mapping the location of inserts, relative to deduced open reading frames, in a population of growing bacteria. A pilot experiment using genomic DNA from a small AT-Cm insertional mutant library (˜5000 inserts) was ‘spiked’ with known quantities of metE mutant DNA and used as a template for PCR and Southern analysis. metE mutant DNA was serially diluted into genomic DNA prepared from the insertional library and these dilutions were used in PCR reactions with a primer pair consisting of one primer specific for AT-Cm (see Methods) and another primer specific for the 5′ coding sequence of metE (FIG. 3). This primer combination (‘insert anchored’ primers) was ˜10⁴ fold more sensitive for detecting the metE insertions from the mixed template than ‘ORF specific’ primers: PCR primer pairs that spanned the coding region of metE (data not shown). PCR reactions using the serially diluted templates were separated by agarose gel electrophoresis, transferred to a nylon membrane and probed with a ³³P-random labeled AT-Cm probe. The results show a significant signal from as few as ˜10 copies of metE insert DNA in a background of ˜10⁷ wild type metE genes (FIG. 3, lane 7).

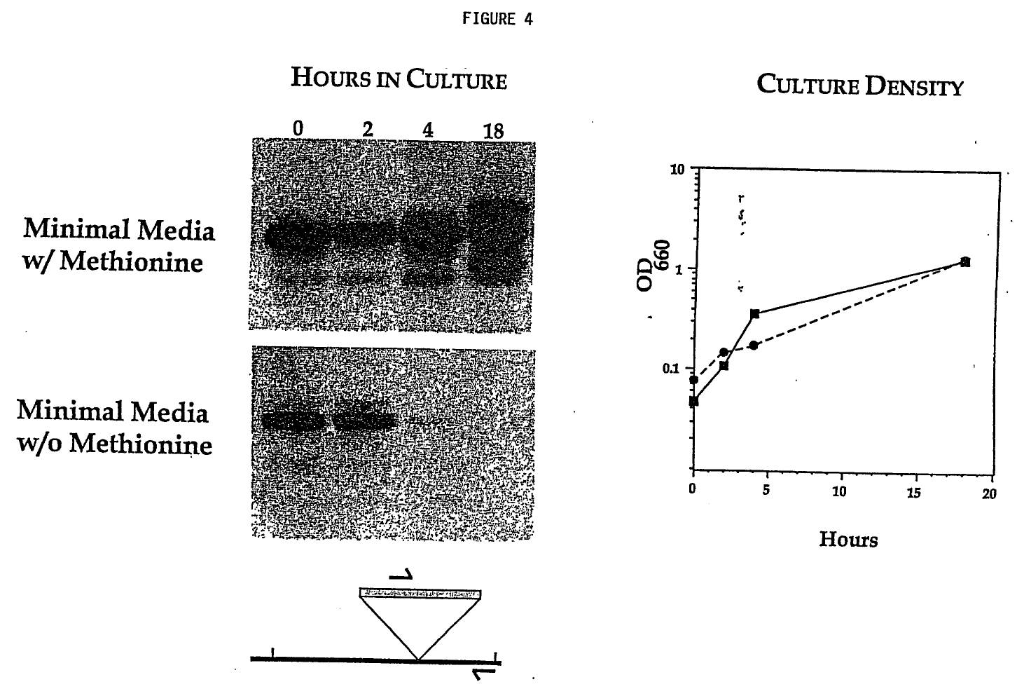

When only genomic DNA from the insertional library was used as a PCR template, we observed several Southern hybridizing bands (FIG. 3, lane 10) with metE specific, insert anchored primers. This result was interpreted as evidence for AT-Cm insertions in metE that are present in the mutant library. These ‘endogenous’ inserts can be detected by PCR and Southern analysis in the presence of small numbers of competing metE mutant DNA templates (FIG. 3, lanes 5, 6 and 8). As the ratio of ‘endogenous’ mutants to metE mutant DNA decreases, the signal from the library diminishes (FIG. 3, lanes 9-4). In order to identify chromosomal insertions, a combination of PCR and Southern analysis gave the required sensitivity and specificity: PCR and agarose gel/ethidium staining alone did not give reliable or reproducible results (data not shown). As the positions of the PCR primers are precisely known (for both the AT-Cm cassette and the ORF of interest), the size of the Southern hybridizing fragments relates to the position of the insert relative to the open reading frame specific primer; thereby identifying the chromosomal location of every insert. By varying the ORF specific primer, a map of the locations of AT-Cm inserts relative to every open reading frame in H. influenzae can be derived. This mapping approach can be used to identify essential genes. ‘Zero time’ analysis. The in vitro transposition reaction can create insertional mutations in both essential and non-essential genes: potentially lethal events will only be manifest after transformation and subsequent expression. Inserts in essential genes will therefore be present in vitro (‘zero time’), and should be lost from the population as the transformation culture grows. This hypothesis was tested using the defined metE mutant and a small AT-Cm insertional library. A culture in complete media (sBHI) was seeded with the metE insert strain and with the small insertional library. This mixed culture was grown for 2 hours and the bacteria were then diluted into minimal media containing all required amino acids or a defined media lacking methionine (Herriott, R. M., et al., “Defined Media for Growth of Haemophilus influenzae”, J. Bacteriol., 101:513-516, (1970); Southern, E. M., “Detection of Specific Sequences Amoung DNA Fragments Separated by Gel Electrophoresis”, J. Mol. Biol., 98:503-517 (1975)). Aliquots at the time of dilution (zero time) and 2, 4 and 18 hours post dilution, were removed and processed for PCR and Southern analysis (FIG. 4). The presence of the metE mutant strain in the culture can be deduced from the insert anchored derived Southern hybridizing band that is clearly visible at the beginning of the experiment (FIG. 4, both panels, lane t=0). The metE insert strain persists throughout the growth of the culture in the samples derived from the minimal media containing methionine, (FIG. 4, upper panel). The samples from minimal media lacking methionine clearly show the disappearance of the metE mutant strain over time (FIG. 4, lower panel). Under the conditions of the experiment, metE is an essential function and cells bearing inserts in this gene are lost from the population. This loss is specific to a subset of mutants, as the growth rate and final cell density of the cultures in both media (with and without methionine) are essentially identical (FIG. 4, graph). The presence of the additional Southern hybridizing bands seen in the minimal media with methionine t=18 hr time point were interpreted as evidence for the outgrowth of ‘endogenous’ metE mutants present in the insertional library. These mutants were identified previously (see FIG. 3, lane 10). As expected, these Southern hybridizing bands derived from the insertional library mutants are not seen in the experimental samples derived from minimal media lacking methionine. These data illustrate the ability to monitor the loss of specific insertional mutants in a growing population of cells, thus providing experimental proof of the essential gene hypothesis.

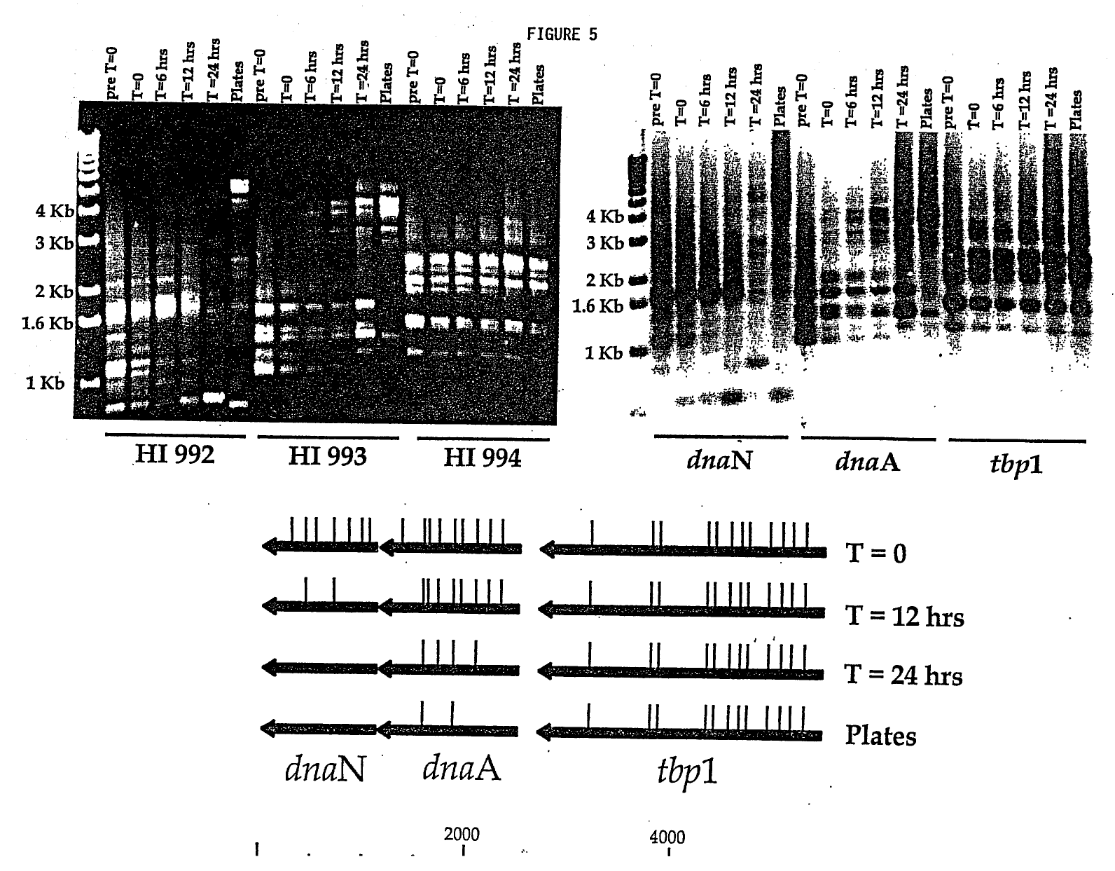

Mutation exclusion. Our definition of gene essentiality states that inserts in essential functions will be lost from a growing population of bacteria. Mapping the positions of AT-Cm inserts in a large mutant library should identify regions of the chromosomes that do not contain inserts: AT-Cm cassettes will be ‘excluded’ from regions of the chromosome required for bacterial survival. Using PCR and Southern analysis to map inserts in a large mutant library (˜40,000 inserts, ˜20 inserts/gene) we examined a contiguous region of the H. influenzae genome, open reading frame by open reading frame, for genes that do not contain AT-Cm inserts. Genomic DNA isolated from the insertional library was used as a template for insert anchored PCR. Each reaction contained a primer pair consisting of a primer specific for AT-Cm and a primer specific for an open reading frame. For ease of analysis, the ORF specific primers were chosen from a single strand of the chromosome. The ethidium stained (FIG. 5, panel A) and resulting Southern analysis (FIG. 5, panel B) was generated from these reactions. The position of the AT-Cm inserts relative to the deduced ORFs in this region of the H. influenzae chromosome were mapped by calculating the size of the Southern hybridizing bands in each lane and are shown above the ORF map (FIG. 5, vertical bars). There are clearly regions that do not contain AT-Cm inserts: these areas map to both annotated and hypothetical open reading frames. When the insert library was examined with PCR primers designed to map AT-Cm inserts present in the opposite orientation, the pattern of AT-Cm insertions in this region of the chromosome was preserved (data not shown). We interpret gaps in the AT-Cm insertion mapping data, which correspond to deduced open reading frames, as defining essential genes. Under these experimental conditions, ORFs 993, 996, 997 and 998 have no At-Cm insertions and are therefore essential, while ORFs 992, 994, 995 clearly have insertions distributed throughout their length and are dispensable genes. This analysis can be continued for every deduced open reading frame in the H. influenzae genome for which a PCR primer can be synthesized.

By placing the insert anchored PCR reactions in sequential order on the gel and manipulating the PCR conditions for longer extensions, overlapping insert mapping data can be generated. Thus, Southern hybridizing bands near the top of the gel in each lane represent AT-Cm inserts in the preceding ORF. This is mostly clearly seen in the repeated pattern of bands in lanes 996, 997 and 999. This kind of analysis provides the precision and confidence required to correctly map the location of the chromosomal AT-Cm insertions.

A method of identifying regions of the H. influenzae chromosome that are required for viability, making use of an in vitro transposition reaction, complete and accurate genomic sequence data and the sensitivity of PCR and Southern analysis to map the chromosomal locations of a selectable marker is the subject of this invention. This approach is generally applicable, though the efficiency of transformation, the accuracy of the genomic sequence and the number of generated insertions will modulate the confidence in the results. Organisms that are naturally competent and whose genome sequence are available, are clear candidates for extending this technique (e.g. Streptococcus pneumoniae, Helicobacter pylori, Neisseria sp.).

The invention lies primarily in the identification of essential open reading frames (“ORF's”) in H. influenzae. These essential ORF's could be discovered with a library of sufficient ‘coverage’ and appropriate genomic PCR primers. They are clearly biologically important, but, until now, they are not generally regarded as primary anti-bacterial drug targets.

The number of inserts we observe in individual ORFs by PCR and Southern analysis corresponds well with our estimate of the number of mutants obtained from colony counting (assuming ˜1000 bp/open reading frame, random insertions and 1.8×10⁶ bp/genome). In analyzing several regions of the H. influenzae chromosome for essential genes, it was noted that the distribution of insert orientation is not random and appears to be influenced by the local DNA transcriptional environment. This is interpreted (and the observation that the number of antibiotic resistant colonies recovered after in vitro transposition is strongly dependent on the chloroamphenicol concentration; higher [chloroamphenicol]=fewer mutants) as evidence that the chloroamphenicol acetyl-transferase (CAT) promoter in AT-Cm is only weakly transcribed in H. influenzae. A weak CAT promoter will reduce possible polar effects of transposase generated insertions on surrounding chromosomal genes, simplifying our analysis.

Mutation exclusion analysis of HI#991-999 identifies a known essential gene, dnaA (HI#993) (Donachie W. D., “The cell cycle of Escherichia coli”, Annu. Rev. Microbiol., 47:199-230, (1993); Marians, K. J., “Replication Fork Propagation”, In F. C. Neidhardt, R. Curtiss, J. L. Ingraham, C. C. Lin, K. B. Low, B. Magasanik, W. S. Reznikoff, M. Riley, M. Schaechter and H. E. Umbarger (ed.) Escherichia coli and Salmonella typhimurium: Cellular and Molecular Biology, vol. 1. American Society for Microbiology, Washington, D.C., p. 750-763 (1996)) and several new essential gene candidates. It was anticipated that dnaN (HI#992) would also be devoid of AT-Cm inserts, but insertions in the 5′ and 3′ regions of this gene were consistently found. The central region of dnaN remained devoid of insertions. Zero time analysis of this gene clearly showed inserts along the entire length of dnaN immediately after transformation, however mutations in the central third of the gene were never seen after selection on solid media, perhaps defining a protein domain required for viability. The unannoted genes HI#996, 997 and 999 are also essential by our analysis: they do not contain At-Cm insertions. HI#998 (ribosomal protein L34) was not directly tested, but inserts in this gene would have been revealed by the overlapping PCR reactions specific for HI#999 (and by exclusion analysis using ORF specific primers derived from the opposite chromosomal strand for HI#997). As expected, this gene is also essential. The transferrins binding proteins (HI#994, 995) are clearly dispensable in rich media, though in an iron limiting environment or in an animal host, these mutants might be non-viable and H. influenzae strains bearing At-Cm inserts in these genes might disappear from the population (Cornelissen, C. N. and P. F. Sparling. “Iron piracy: acquisition of transferrin-bound iron by bacterial pathogens”, Mol. Microbiol., 14:843-850 (1994)).

It is anticipated that using this mutant library, and searching for genes required for survival in animal models of infection, virulence determinants could be identified as well. This approach could be refined further, to identify genes required for survival in specific niches or organs (e.g. lung vs. liver vs. spleen) or in different animal models of infection (e.g. murine vs. rat). Given the size of the mutant libraries we can now generate, it is believed that genome scanning could give a more complete picture of the functions required for pathogenesis than other in vivo mutagenesis methods (Mahan, M. J., et al., “Antibiotic-based selection for bacterial genes that are specifically induced during infection of a host”, Proc. Natl. Acad Sci. USA., 92(3):669-73, (1995); Mei, J. M., et al., “Identification of Staphylococcus aureus virulence genes in a murine model of bacteraemia using signature-tagged mutagenesis”, Mol. Microbiol., 26:399-407, (1997)). An important achievement would be to generate a list of essential genes required for bacterial viability. As a matter of convenience, rich media (sBHI) was chosen as a growth condition for selection. The selective properties of solid media vs. broth culture were noted in initial experiments, and sBHI-agar was chosen for generating the mutant libraries. Other culture conditions could be tested, including various minimal media, partial oxygen pressure, heat shock, cold shock, growth in serum, limiting iron, etc. Identifying functions required for survival in stationary phase could also be considered.

Several different approaches to identifying essential genes in microorganism have been proposed, both before and after the availability of genomic sequences (Schmid, M. B., et al., “Genetic analysis of temperature-sensitive lethal mutants of Salmonella typhimurium”, Genetics, 123:625-33, (1989)). Post-genomic approaches include a systematic ‘knock-out’ strategy, being undertaken by the yeast community, ‘in silico’ analysis to determine common, shared and unique open reading frames (Arigoni, F., et al., “A genome based approach for the identification of essential bacterial genes”, Nature Biotech., 16:851-856,(1998)), systematic complementation of temperature sensitive alleles and a similar in vitro transposition mutagenesis strategy that has recently been described in “Systematic Identification of essential genes by in vitro mariner mutagensis”, herein incorporated by reference Akerley, B. J., et al., “Systematic Identification of essential genes by in vitro mariner mutagensis”, Proc. Natl. Acad. Sci. USA., 95:8927-8932, (1998). The present inventors have developed and used a well characterized in vitro transposition system to generate a large mutant insert library and analyzed the library by mapping the location of inserts relative to open reading frames and by monitoring the rate of loss of particular mutants. The ability to follow the disappearance of a particular mutant over time provides both a positive control for the ORF of interest (that the in vitro transposition reaction targeted the ORF) and biological information concerning the open reading frame itself. The rate of gene loss will be modulated by a number of factors, including the steady state level of expression of the protein, its the half life, the cell doubling time and the cellular function that is abrogated. This additional data will be relevant to choosing targets for anti-bacterial drug discovery.

Recently, specific regions of H. influenzae have been targeted by in vitro transposition mutagenesis by using ˜15 kbp genomic fragments, generated by long PCR, as templates. In these ‘focused libraries’ we can obtain 10,000 mutants, roughly 1 insert/1.5 bp, making a truly saturated mutant library. The recognition sequence for Ty-1 is four basepairs, allowing for simple and efficient construction of translational fusions for structure/function studies. This, coupled with the focused mutant library approach, would allow for detailed basepair by basepair topological analysis (using alkaline phosphatase fusions (Manoil C. and J. Beckwith, “A genetic approach to analyzing membrane protein topology”, Science, 233:1403-1408, (1986)) and protein functional domain identification (as loss of an enzymatic function could be rapidly correlated to the position of inserts). This facile system could also be used to generate transcriptional fusions with reporter genes (e.g. GFP or 62-galactosidase) for cell sorting and identification.

Genome scanning provides an experimental technique for assigning a rudimentary annotation to the large fraction of bacterial genomes that have no known function. This method, and its variations, will provide solutions to understanding and predicting the minimal gene complement required for autonomous bacterial survival.

1. An essential bacterial gene comprising a purified polynucleotide isolated from Haemophilus influenzae, wherein said polynucleotide has at least 70% identity with a sequence selected from the group consisting of SEQUENCE ID NOS 1, 3, 5, 7, 9, 11, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127 and 129 and fragments or complements thereof, wherein said polynucleotides are essential to said Haemophilus influenzae's survival.

2. The polynucleotide of claim 1, wherein said polynucleotide selectively hybridizes to a nucleic acid sequence selected from the group consisting of SEQUENCE ID NOS 1, 3, 5, 7, 9, 11, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127 and 129 and fragments or complements thereof.

3. The polynucleotide of claim 2, wherein said polynucleotide has an overall length of about 20 to about 50 nucleotides.

4. The polynucleotide of claim 2, wherein said polynucleotide has an overall length of about 10 to 25 nucleotides.

5. The polynucleotide of claim 2, wherein said polynucleotide is produced by recombinant techniques.

6. The polynucleotide of claim 2, wherein said polynucleotide is produced by synthetic techniques.

7. A recombinant expression system comprising a nucleic acid sequence that includes an open reading frame, wherein said open reading frame is operably linked to a control sequence compatible with a desired host, and said nucleic acid sequence has at least 50% identity with a sequence selected from the group consisting of SEQUENCE ID NOS 1, 3, 5,7,9, 11, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127 and 129 and fragments or complements thereof.

8. A cell transfected with the recombinant expression system of claim 7.

9. A polypeptide having at least 50% identity with an amino acid sequence selected from the group consisting of SEQUENCE ID NOS. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, and 130 and fragments thereof, wherein said polypeptide is essential to Haemophilus influenzai's survival.

10. The polypeptide of claim 9, wherein said polypeptide is produced by recombinant techniques.

11. The polypeptide of claim 10, wherein said polypeptide is produced by synthetic techniques.

12. A method of determining whether a gene is essential to a bacterium's survival, said method comprising:
mutagenizing bacterial cells by integrating a transposon in the genome of said cells;
identifying the insertion sites of said transposon; and
correlating the insertion site with the survival or death of said bacterial cell wherein the death of said cell correlates with the gene said transposon was inserted into as being essential.

13. The method of claim 12 wherein the transposon is inserted into a gene selected from the group consisting of DEQ. ID. NOS. 1, 3, 5, 7, 9, 11, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, and 129.

14. A method for screening substances to determine those substances which function to inhibit essential Haemophilus influenzae polypeptides, said method comprising: contacting a polypeptide product selected from the group consisting of SEQUENCE ID NOS. 2, 4, 6, 8, 10, 12, 14, 16, 18, 20, 22, 24, 26,28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, and 130, with substance of interest; and measuring the response.

15. The method of claim 14 wherein said measurement step is conducted by a screen selected from the group consisting of a specific screen, enzyme screen, general screen, affinity screen, phenotypic screen and binding screen.

16. A lethal method of eliminating Haemophilus ionfluenzae comprising: altering the polynucleotide sequences selected from the group consisting of SEQ. ID. NOS. 1, 3, 5, 7, 9, 11, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, and 129; wherein said altering step is selected from the group consisting of nucleic acid deletions, substitutions, or insertions.

17. A lethal method of eliminating Haemophilus influenzae comprising: altering the amino acid sequences selected from the group consisting of SEQ. ID. NOS. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, and 130; wherein said altering step is selected from the group consisting of amino acid deletions, substitutions, or insertions.
